(19) United States
(12) Patent Application Publication
(10) Pub. No.: US 2024/0316364 A1
(43) Pub. Date: Sep. 26, 2024

(54) QUALITY ASSURANCE METHOD FOR RADIOTHERAPY DEVICE, COMPUTER DEVICE, AND STORAGE MEDIUM

(71) Applicant: SHANGHAI UNITED IMAGING HEALTHCARE CO., LTD., SHANGHAI (CN)

(72) Inventors: ZHONG-HUA SHI, SHANGHAI (CN); CAN LIAO, SHANGHAI (CN)

(21) Appl. No.: 18/611,891

(22) Filed: Mar. 21, 2024

(30) Foreign Application Priority Data
Mar. 21, 2023 (CN) 202310282179.3

(51) Int. Cl. A61N 5/10

(52) U.S. Cl. CPC A61N 5/1075; A61N 5/1077; A61N 2005/1054; A61N 2005/1076

(57) ABSTRACT

A quality assurance (QA) method for a radiotherapy device, a QA device for a radiotherapy device, and a computer device. The radiotherapy device includes an electronic portal imaging device (EPID). The QA method for the radiotherapy device includes: obtaining an image sequence based on acquirement of EPID, the image sequence comprising one or more correction images for correcting the EPID, and one or more portal images for a QA object; and determining a QA result of the QA object of the radiotherapy device according to a calibration parameter corresponding to the one or more correction images and according to the one or more portal images.

### CROSS-REFERENCE TO RELATED APPLICATIONS

The present application claims the priority of the Chinese patent application No. 202310282179.3, filed on Mar. 21, 2023, and entitled “Quality Assurance Method for Radiotherapy Device, Quality Assurance Device for Radiotherapy Device, Computer Device, and Storage Medium”, which is incorporated herein by reference in its entirety.

### TECHNICAL FIELD

The present application relates to the field of radiotherapy technology, and in particular, to a quality assurance (QA) method for a radiotherapy device, a QA device for a radiotherapy device, a computer device, a storage medium, and a computer program product.

### BACKGROUND

Nowadays, radiotherapy technology plays an increasingly important role in tumor treatment. Precision radiotherapy is the key to ensure the treatment effect. In order to improve the accuracy of radiotherapy, it is necessary to conduct a QA on each moving part of the radiotherapy device, i.e., each QA object, to ensure that the radiotherapy device operates in good condition.

However, in related technologies, third-party devices are often required when a QA is performed on a radiotherapy device. Since the introduction of the third-party devices will affect the measurement results of the QA objects of the radiotherapy device, it will ultimately affect the QA accuracy of each QA object of the radiotherapy device.

### SUMMARY

The present disclosure provides a QA method for a radiotherapy device, a QA device for a radiotherapy device, a computer device, a non-non-transitory computer-readable storage medium, and a computer program product.

On a first aspect, the present disclosure provides a QA method for a radiotherapy device. The radiotherapy device includes an electronic portal imaging device (EPID). The method includes the following steps. An image sequence based on acquirement of EPID is obtained, the image sequence includes one or more correction images for correcting the EPID, and one or more portal images of a QA object. A QA result of the QA object of the radiotherapy device is determined according to a calibration parameter corresponding to the one or more correction images and according to the one or more portal images of the QA object.

In one of the embodiments, before obtaining the image sequence based on the acquirement of EPID, the QA method further includes the following steps. A QA plan sequence of the radiotherapy device is obtained, the QA plan sequence includes a QA plan for the QA object of the radiotherapy device. The QA plan for the QA object is executed.

In one of the embodiments, the QA plan for the QA object comprises setting a shape of a radiation field and a position of a QA phantom.

In one of the embodiments, determining the QA result of the QA object for the radiotherapy device according to the calibration parameter corresponding to the one or more correction images and according to the one or more portal images, includes: determining current measurement data corresponding to the QA object according to the calibration parameter corresponding to the one or more correction images, and according to the one or more portal images, and determining the QA result of the QA object of the radiotherapy device based on the current measurement data corresponding to the QA object.

In one of the embodiments, the calibration parameter comprises a pose calibration parameter corresponding to the EPID. Determining the current measurement data corresponding to the QA object according to the calibration parameter corresponding to the one or more correction images, and according to the one or more portal images includes: calibrating the portal images according to the pose calibration parameter, to obtain a target portal image of the QA object, and determining the current measurement data corresponding to the QA object based on image features of the target portal image.

In one of the embodiments, the QA object comprises an isocenter, and the target portal image corresponding to an isocenter measurement includes a phantom image of a QA phantom at each preset gantry rotation angle and a square field image formed by a beam-limiting system at each preset gantry rotation angle, or a phantom image of a QA phantom in a corresponding field at each preset gantry rotation angle. The QA phantom includes multiple mark points. Determining the current measurement data corresponding to the QA object based on the image features of the target portal image includes: determining a projection matrix corresponding to each preset gantry rotation angle according to mark-point spatial coordinates of the multiple mark points at initial positions, and according to mark-point projection coordinates of the multiple mark points in each phantom image, determining a straight-line trail of a radiation beam axis in a spatial coordinate system corresponding to the radiation beam axis at each preset gantry rotation angle according to the projection matrix corresponding to each preset gantry rotation angle, and according to projection coordinates of a radiation field center corresponding to the radiation field center in the square field image under each preset gantry rotation angle, and determining a radiation isocenter parameter corresponding to an intersection point of straight-line trails of radiation beam axes.

In one of the embodiments, the QA object includes a table, and the target portal image corresponding to the table includes a first initial phantom image and first post-movement phantom images acquired at a gantry rotation angle, and the first post-movement phantom images include phantom images each corresponding to a direction of each coordinate axis of a spatial coordinate system. The QA phantom includes multiple mark points.

Determining the current measurement data corresponding to the QA object based on the image features of the target portal image includes: determining post-movement mark-point projection coordinates of the multiple mark points in coordinate axes according to each of the first post-movement phantom images, determining actual movement parameters of the table in the coordinate axes respectively corresponding to the gantry angle and the post-movement mark-point projection coordinates.

In one of the embodiments, the QA object includes a gantry, the target portal image corresponding to the gantry includes a second initial phantom image and second post-movement phantom images of the QA phantom, and the second post-movement phantom image includes phantom images at different heights of the table.

Determining the current measurement data corresponding to the QA object according to the image features of the target portal image, includes: determining, for the same mark point, target mark-point projection coordinates in the second initial phantom image and in each of the second post-movement phantom images to obtain the current measurement data corresponding to the gantry.

In one of the embodiments, the QA object comprises a beam component, and the target portal image corresponding to the beam component comprises a preset number of square field images.

Determining the current measurement data corresponding to the QA object based on the image features of the target portal image, includes: superimposing the preset number of square field images to obtain a superimposed square field image, translating the superimposed square field image and adjusting the translated square field image according to an image scaling coefficient, to obtain the current measurement data corresponding to the beam component.

In one of the embodiments, the QA object includes a collimator, and the target portal image corresponding to the collimator comprises a first correction square field rotation image corresponding to a first collimator angle and a second correction square field rotation image corresponding to a second collimator angle.

Determining the current measurement data corresponding to the QA object according to the image features of the target portal image, includes: detecting a boundary of a jaw according to the first correction square field rotation image and the second correction square field rotation image, to obtain a first boundary line and a second boundary line; determining a first boundary measurement inclination angle and a second boundary measurement inclination angle according to the first boundary line and the second boundary line; and obtaining the current measurement data corresponding to the collimator according to the first boundary measurement inclination angle and the second boundary measurement inclination angle.

In one of the embodiments, the image sequence comprising a plurality of correction images. The plurality of correction images comprise a correction open filed image, a correction comb-shaped beam image, and a correction square field image. The correction square field image comprises a correction square field rotation image at each preset collimator angle.

Before determining the QA result of the QA object according to the calibration parameter corresponding to the one or more correction images, and according to the one or more portal images, the method further includes: calibrating the correction open filed image to obtain a first calibration parameter; determining a second calibration parameter based on leaf description parameters corresponding to leaves of a multi-leaf collimator (MILC) in the correction comb-shaped beam image; fitting a radiation field center corresponding to the correction square field rotation image at each preset collimator angle, to obtain a third calibration parameter; and determining a pose calibration parameter corresponding to the plurality of correction images based on the first calibration parameter, the second calibration parameter and the third calibration parameter.

In one of the embodiments, the QA object comprises the MLC, and the target portal image corresponding to the MLC comprises a collimator portal image, and the collimator portal image is an image acquired after a shape of the radiation field of the MLC is configured to have a first size.

Determining the current measurement data corresponding to the QA object according to the image features of the target portal image includes: performing a transverse gradient transformation on the collimator portal image, and determining a position of a pixel with the largest gray gradient change corresponding to each leaf in the collimator portal image, on which a transverse gradient transformation is performed, as a pixel position to be converted corresponding to each leaf, and converting each pixel position to be converted into a radiation field position in a radiation field coordinate system, to obtain a current measurement position of each leaf, and using the current measurement position of each leaf as the current measurement data corresponding to the MLC.

In one of the embodiments, the QA object includes a jaw, and the target portal image corresponding to the jaw comprises a jaw beam image, and the jaw beam image is an image acquired after a shape of the radiation field of the jaw is configured to have a second size.

Determining the current measurement data corresponding to the QA object according to the image features of the target portal image includes: performing a longitudinal gradient transformation on the jaw beam image, determining a jaw pixel position, and obtaining a longitudinal jaw measurement position according to the jaw pixel position; performing a transverse analysis on the jaw beam image to obtain a transverse jaw measurement position; and using the longitudinal jaw measurement position and the transverse jaw measurement position as the current measurement data corresponding to the jaw.

In one of the embodiments, the QA object includes the plate detector. Determining the current measurement data corresponding to the QA object according to the image features of the target portal image, includes: determining a current measurement position of the collimator rotation center on a plate coordinate system, where the plate coordinate system is a pixel coordinate system corresponding to the plate detector; determining a leaf lateral width of the MLC based on the correction comb-shaped beam image, and determining a distance from the plate detector to an X-ray source of the radiotherapy device based on the leaf lateral width as a current measured SID; and obtaining the current measurement data corresponding to the plate detector based on the current measurement position of the collimator rotation center in the plate coordinate system and based on the current measured SID.

The present disclosure further provides a QA method for a radiotherapy device, the radiotherapy device includes an electronic portal imaging device (EPID). The QA method for the radiotherapy device includes: determining a calibration parameter corresponding to one or more correction portal images based on the one or more correction images for calibrating the EPID acquired by the EPID; obtaining a target portal image of the QA object, based on the calibration parameter and one or more portal images acquired by the EPID corresponding to the QA object; and determining a QA result of the QA object of the radiotherapy device according to the target portal image.

The present disclosure further provides a QA device for a radiotherapy device. The device includes an obtaining module and a result determining module.

The obtaining module is configured to obtain an image sequence based on acquirement of EPID. The image sequence includes one or more correction images configured to correct the EPID, and one or more portal images of a QA object.

The result determining module is configured to determine a QA result of the QA object for the radiotherapy device according to a calibration parameter corresponding to the one or more correction images and according to the one or more portal images.

The present disclosure further provides a computer device. The computer device includes a memory and a processor. The memory stores a computer program. When the processor executes the computer program, it implements the following steps: obtaining an image sequence based on acquirement of EPID, the image sequence comprising one or more correction images for correcting the EPID, and one or more portal images of a QA object, and determining a QA result of the QA object of the radiotherapy device according to a calibration parameter corresponding to the one or more correction images and according to the one or more portal images.

The present disclosure further provides a non-transitory computer-readable storage medium. The computer-readable storage medium has executable instructions stored thereon. When the executable instructions are executed by the processor, the following steps are implemented: obtaining an image sequence based on acquirement of EPID, the image sequence comprising one or more correction images for correcting the EPID, and one or more portal images of a QA object, and determining a QA result of the QA object of the radiotherapy device according to a calibration parameter corresponding to the one or more correction images and according to the one or more portal images.

The present disclosure further provides a computer program product. The computer program product includes executable instructions. When the executable instructions are executed by a processor, the following steps are implemented.

An image sequence based on acquirement of EPID is obtained. The image sequence includes correction images for correcting the EPID, and portal images of a QA object. A QA result of the QA object of the radiotherapy device is determined according to a calibration parameter corresponding to the one or more correction images and according to the one or more portal images.

According to the above-mentioned QA method for the radiotherapy device, the QA device for the radiotherapy device, the computer device, the storage medium, and the computer program product, the image sequence is obtained based on acquirement of EPID. The image sequence includes one or more correction images for correcting the EPID, and one or more portal images of a QA object. A QA result of the QA object of the radiotherapy device are determined according to a calibration parameter corresponding to the one or more correction images and according to the one or more portal images.

In this way, the one or more correction images and the one or more portal images are obtained based on the acquirement of the EPID. According to the calibration parameter corresponding to the correction images, the impact of the position change of the EPID on each QA object can be removed. Based on the calibration parameter corresponding to the correction images and the portal images, the state of each QA object can be accurately analyzed, the QA result corresponding to the radiotherapy device can be accurately determined, and the problem that using a third-party device for QA of radiotherapy device affects the measurement results of the QA object can be solved. While realizing an automatic QA of the radiotherapy device, the invention can effectively improve the QA accuracy of the radiotherapy device.

### BRIEF DESCRIPTION OF THE DRAWINGS

FIG. 1 is a schematic flow diagram of a QA method for a radiotherapy device according to an embodiment of the present disclosure;

FIG. 2 shows a correction open filed image according to an embodiment of the present disclosure;

FIG. 3 shows a correction comb-shaped beam image according to an embodiment of the present disclosure;

FIG. 4 shows correction square field rotation images according to an embodiment of the present disclosure;

FIG. 5 shows phantom images and square field images according to an embodiment of the present disclosure;

FIG. 6 shows straight-line trails of radiation beam axes according to an embodiment of the present disclosure;

FIG. 7 shows projections of the straight-line trails of the radiation beam axes on the XZ plane according to an embodiment of the present disclosure;

FIG. 8(a) shows a first initial phantom image according to an embodiment of the present disclosure;

FIG. 8(b) shows a phantom image corresponding to an x-direction offset according to an embodiment of the present disclosure;

FIG. 8(c) shows a phantom image corresponding to a y-direction offset according to an embodiment of the present disclosure;

FIG. 8(d) shows a phantom image corresponding to a z-direction offset according to an embodiment of the present disclosure;

FIG. 8(e) shows an ISO-angle offset phantom image according to an embodiment of the present disclosure;

FIG. 9(a) shows a second initial phantom image according to an embodiment of the present disclosure;

FIG. 9(b) shows a second post-movement phantom image according to an embodiment of the present disclosure;

FIG. 9(c) shows a second post-movement phantom image according to another embodiment of the present disclosure;

FIG. 9(d) shows an intersection point of scaling paths of projection positions corresponding to mark points in superimposed phantom images according to an embodiment of the present disclosure;

FIG. 10 shows changes of positions of the light source points under different preset gantry rotation angles according to an embodiment of the present disclosure;

FIG. 11 is a schematic view of shifting a position of the radiation field center to a position of a plate center according to an embodiment of the present disclosure;

FIG. 12 shows a reference square field image, a target square field image and a ratio image according to an embodiment of the present disclosure;

FIG. 13(a) shows first fit boundary lines in a first correction square field gradient image according to an embodiment of the present disclosure;

FIG. 13(b) shows second fit boundary lines in a second correction square field gradient image according to an embodiment of the present disclosure;

FIG. 14 is a schematic view showing positions of central pixel rows where centers of leaves are located respectively in a comb-shaped beam image according to an embodiment of the present disclosure;

FIG. 15(a) shows a radiation field coordinate system in a collimator portal image according to an embodiment of the present disclosure;

FIG. 15(b) shows a collimator portal image on which a transverse gradient transformation is performed according to an embodiment of the present disclosure;

FIG. 16 shows a radiation field coordinate system in a jaw beam image according to an embodiment of the present disclosure;

FIG. 17 shows a U/V coordinate system corresponding to a plate detector according to an embodiment of the present disclosure;

FIG. 18 is a schematic flow diagram of a QA method for a radiotherapy device according to another embodiment of the present disclosure;

FIG. 19 is a schematic flow diagram of a QA method for a radiotherapy device according to another embodiment of the present disclosure;

FIG. 20 is a block diagram showing a structure of a QA device for a radiotherapy device according to an embodiment of the present disclosure; and

FIG. 21 is a schematic view showing an internal structural configuration of a computer device according to an embodiment of the present disclosure.

### DETAILED DESCRIPTION OF THE EMBODIMENTS

In order to make the purpose, technical solutions and advantages of the present disclosure clearer, the present disclosure will be further described in detail hereinafter with reference to the drawings and embodiments. It should be understood that the specific embodiments described herein are only used to explain the present disclosure but are not intended to limit the present disclosure.

It should be noted that the terms “first”, “second”, etc. in the description and claims of the present disclosure and the above-mentioned drawings are used to distinguish similar objects and are not necessarily used to describe a specific order or sequence. It should be understood that the data so used are interchangeable under appropriate circumstances so that the embodiments of the disclosure described herein may be practiced in sequences other than those illustrated or described herein. The implementations described in the following exemplary embodiments do not represent all implementations consistent with the present disclosure. Rather, they are merely examples of apparatus and methods consistent with aspects of the disclosure as detailed in the appended claims.

In related technologies, a quality assurance (QA) of a radiotherapy device is mainly performed through third-party devices. For example, a movement accuracy and repeatability of a multi-leaf collimator (MLC) and of a jaw may be measured by a water tank and a film. A rotation accuracy and repeatability of a collimator and a gantry may be measured by an inclinometer. A movement accuracy and repeatability of an electronic portal imaging device (EPID) and a table may be measured by a dial indicator or a laser range finder, an isocenter may be measured by a front pointer or a Weisfeiler-Lehman (WL) method, and a beam may be measured by an electrometer. Since the introduction of the third-party devices will affect measurement results of the QA objects of the radiotherapy device, and ultimately affect the QA accuracy of each QA object of the radiotherapy device, in related technologies, there is a problem of poor accuracy of QA of the radiotherapy device.

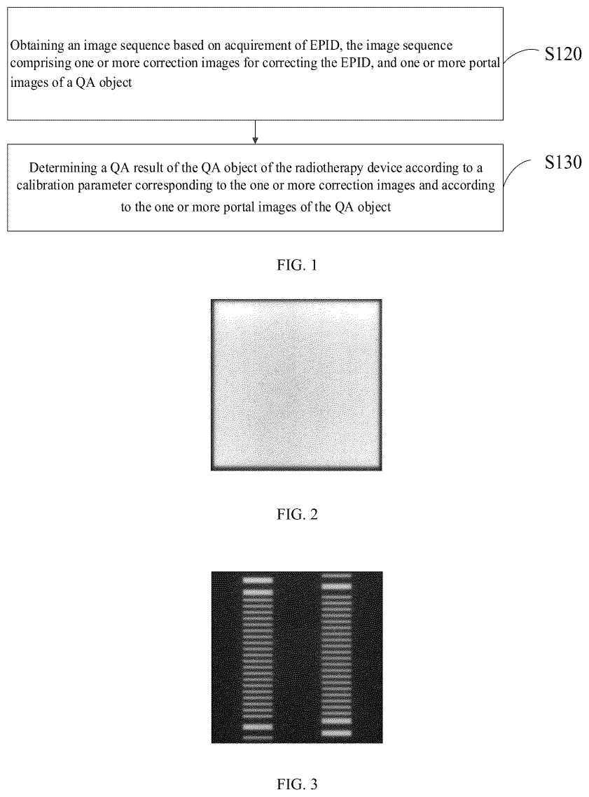

In response to the above technical problems, in one of the embodiments of the present disclosure, as shown in FIG. 1, a QA method for a radiotherapy device is provided. In this embodiment, the method is described by taking the method applied to the radiotherapy device itself as an example. In this embodiment, the radiotherapy device includes an electronic portal imaging device (EPID), and the QA method for the radiotherapy device includes the following steps S120 and S130.

In step S120, an image sequence is obtained based on acquirement of EPID. The image sequence includes one or more correction images for correcting the EPID, and one or more portal images of a QA object.

The EPID of the radiotherapy device is prone to geometric deformation, and the mechanical position of the radiotherapy device is prone to offset. In a specific implementation, in order to remove the impact of the geometric deformation and the position offset of the EPID on the measurement results, in the QA method for the radiotherapy device based on the EPID of this disclosure, the image sequence is obtained by including one or more correction images and one or more portal images of the QA object. The one or more correction images are acquired by the EPID to correct the EPID, and the one or more portal images of the QA object are acquired by the EPID to perform a QA on the QA object.

In step S130, a QA result of the QA object of the radiotherapy device is determined, according to a calibration parameter corresponding to the one or more correction images, and the one or more portal images for the QA object.

In a specific implementation, the radiotherapy device may determine the calibration parameter corresponding to the EPID based on the correction images, and determine the QA result of the QA object of the radiotherapy device based on the calibration parameter and the portal images.

In the QA method for the radiotherapy device of the present disclosure, the image sequence acquired based on the EPID. The image sequence includes the correction images configured to correct the EPID, and the portal images. The QA result of the QA object of the radiotherapy device is determined according to the calibration parameter corresponding to the correction images and according to the portal images. Because the image sequence includes the correction images configured to correct the EPID, this QA method may calibrate the mechanical position and the geometric deformation of the EPID in real time, to remove the impact of the position change of the EPID on the measurement result of the QA object of the radiotherapy device.

In one of the embodiments of the present disclosure, before step S120 of acquiring the image sequence based on the EPID, the QA method for the radiotherapy device further includes step S100 and step S110.

In step S100, a QA plan sequence of the radiotherapy device is obtained, where the QA plan sequence includes a QA plan for the QA object of the radiotherapy device.

In step S110, the QA plan for the QA object is executed.

The QA plan sequence corresponding to the radiotherapy device includes the QA plan for the QA object of the radiotherapy device.

The QA object of the radiotherapy device may include a multi-leaf collimator (MLC), a jaw, a collimator, an EPID, a gantry, a table, an isocenter, a laser, and/or a beam, etc.

In a specific implementation, the radiotherapy device may obtain the QA plan sequence, which includes the QA plan for the QA object of the radiotherapy device. The radiotherapy device may execute the QA plan for the QA object.

In one of the embodiments of the present disclosure, the QA plan for the QA object includes setting a shape of the radiation field and a position of a QA phantom for the QA object.

The portal image of the QA object is acquired after setting the shape of the radiation field and the position of the QA phantom for the QA object.

In a specific implementation, the radiotherapy device may execute the QA plan for the QA object, and set the shape of the radiation field and the position of the QA phantom for the QA object, according to the QA plan for the QA object.

In one of the embodiments of the present disclosure, step S130 of determining the QA result of the QA object of the radiotherapy device according to the calibration parameter corresponding to the one or more correction images and the one or more portal images for the QA object, includes step S1301 and step S1302.

In step S1301, the current measurement data corresponding to the QA object are determined according to the calibration parameter corresponding to the one or more correction images, and the one or more portal images.

In a specific implementation, the radiotherapy device may determine the calibration parameter corresponding to the EPID according to the one or more correction images, and determine the current measurement data corresponding to the QA object according to the calibration parameter and the one or more portal images.

In step S1302, the QA result of the QA object of the radiotherapy device is determined based on the current measurement data corresponding to the QA object.

In a specific implementation, the radiotherapy device may determine the QA result corresponding to the QA object of the radiotherapy device based on the current measurement data corresponding to the QA object and the corresponding reference data.

In the above QA method of the radiotherapy device, the QA plan sequence corresponding to the radiotherapy device is obtained. The QA plan sequence includes the QA plan for the QA object of the radiotherapy device. The QA plan for the QA object is executed, and the image sequence is acquired based on the EPID of the QA object. The current measurement data corresponding to the QA object are determined according to the calibration parameter corresponding to the one or more correction images, and the one or more portal images. According to the current measurement data corresponding to the QA object, the QA result corresponding to the QA object of the radiotherapy device is determined.

In this way, a specific QA plan sequence corresponding to the radiotherapy device is used. The QA plan sequence includes the QA plan for the QA object of the radiotherapy device, so that the correction images and the portal images may be acquired based on the EPID of the QA object. Based on the calibration parameter corresponding to the correction image, the impact of the position change of the EPID on the measurement result of the QA object may be removed. Thus, the status of each QA object may be obtained through accurate analysis according to the calibration parameter corresponding to the correction images and the portal images, so that the QA result corresponding to the radiotherapy device is determined accurately based on the current measurement data corresponding to the QA object, which solves the problem that the measurement result of the QA object is affected by using the third-party device to perform the QA on the radiotherapy device. While realizing an automatic QA of the radiotherapy device, the method of the disclosure can effectively improve the QA accuracy of the radiotherapy device.

In one of the embodiments, the calibration parameter includes a pose calibration parameter corresponding to the EPID. Step S1301 of determining the current measurement data corresponding to the QA object according to the calibration parameter corresponding to the one or more correction images and the one or more portal images, includes step S1301a and step S1301b. In step S1301a, the one or more portal images are calibrated according to the pose calibration parameter, to obtain a target portal image for the QA object. In step S1301b, the current measurement data corresponding to the QA object are determined, based on the image features of the target portal image.

When the correction images are being acquired, the QA phantom needs to be moved out from the radiation field range of the EPID by moving a table.

In a specific implementation, when the radiotherapy device determines the current measurement data corresponding to each QA object according to the calibration parameter corresponding to the one or more correction images and the one or more portal images, the mechanical deformation of a support structure of the EPID and non-uniformity of the EPID pixel responses have a great impact on the analysis of the measurement data of other QA objects. Therefore, in order to remove the impact of the EPID on the measurement results corresponding to each QA object, the pose calibration parameter corresponding to the EPID needs to be determined based on the one or more correction images, and the one or more portal images needs to be calibrated based on the pose calibration parameter, to obtain the target portal image. Finally, according to the image features of the target portal image, the current measurement data corresponding to each QA object is determined.

In an embodiment, the image sequence comprising a plurality of correction images. The correction images include a correction open filed image, a correction comb-shaped beam image and a correction square field image.

The correction open filed image is an image acquired by the EPID under exposure conditions. In order to facilitate understanding of those skilled in the art, FIG. 2 shows a correction open filed image.

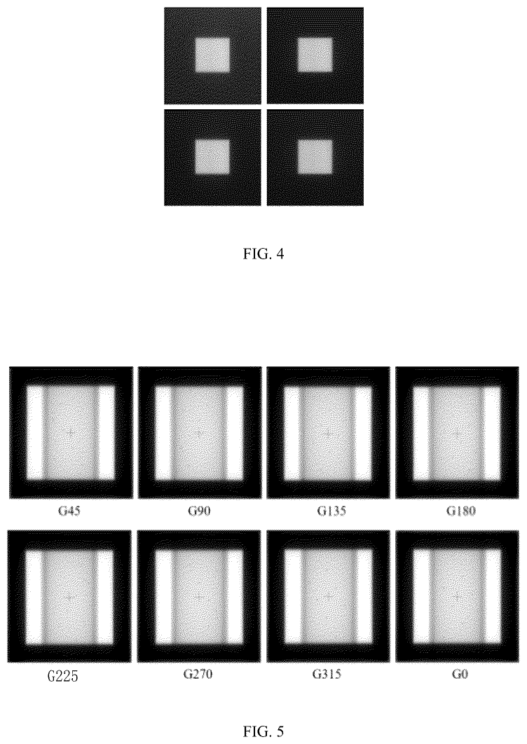

The correction comb-shaped beam image is a portal image acquired by the EPID when components of the MLC alternately extend outwards and retracts to be configured as a comb shape. In order to facilitate understanding of those skilled in the art, FIG. 3 shows a correction comb-shaped beam image.

The correction square field image is a square field image acquired by the EPID when the shape of the radiation field of the MLC and/or the jaw are configured to be square.

The correction square field image includes the correction square field rotation images under preset collimator angles respectively. The intervals between the preset collimator angles are equal. For example, the intervals may be 90°, i.e., the collimator angles are 180°, 90°, 0°, and 270° respectively. The intervals between the preset collimator angles are not limited specifically. In order to facilitate the understanding of those skilled in the art, FIG. 4 shows correction square field rotation images corresponding to the preset collimator angles of 0°, 90°, 180°, and 270° respectively.

In the process of determining the pose calibration parameter corresponding to the EPID based on the correction images by the radiotherapy device, the radiotherapy device may calibrate the correction open filed image to obtain a first calibration parameter corresponding to the EPID. Specifically, the EPID may correct bad pixels in the correction open filed image, which have no response, or whose responses are significantly different from those of other pixels. What's more, the inconsistencies of the responses of pixels in the correction open filed image need to be corrected. For example, the correction open filed image may be normalized, and then all images to be processed subsequently are divided by the normalized open filed image.

In the process of determining the pose calibration parameter corresponding to the EPID based on the correction images by the radiotherapy device, the radiotherapy device may further determine a second calibration parameter corresponding to the EPID, according to leaf description parameters of the leaves of the MLC in the correction comb-shaped beam image. Specifically, the radiotherapy device may determine a position of the central pixel row where a center of each leaf of the MLC is located (i.e., the position of the center of each open bar or dark bar in FIG. 3), determine a leaf width changing from top to bottom or a rotation angle each leaf of the MLC, and determine a projection width of each of leaf the MLC, to obtain the leaf description parameters corresponding to the leaves of the MLC. Thus, in the process of determining the second calibration parameter corresponding to the EPID based on the leaf description parameters corresponding to the leaves of the MLC by the radiotherapy device, the radiotherapy device may calculate a rotation angle of the EPID around each axis which may be configured to horizontally correct the EPID, based on the leaf width changing from top to bottom or based on the rotation angle of the leaf in the correction comb-shaped beam image. The radiotherapy device may determine the image scaling coefficient of the EPID, based on the leaf projection width in the correction comb-shaped beam image. Then the position of the central pixel row where the center of each leaf is located, the rotation angle of the EPID around each axis, and the image scaling coefficient of the EPID may be used as the second calibration parameter corresponding to the EPID.

Furthermore, in the process of determining the pose calibration parameter corresponding to the EPID based on the correction images by the radiotherapy device, the radiotherapy device may further fit a radiation field center corresponding to the correction square field rotation image at each preset collimator angle, to determine the corresponding position of the collimator rotation center on the plate detector of the EPID, and to obtain the third calibration parameter corresponding to the EPID.

In this way, the radiotherapy device may determine the pose calibration parameter corresponding to the correction images, based on the first calibration parameter, the second calibration parameter and the third calibration parameter corresponding to the EPID. Therefore, in the process of calibrating the portal image of the QA object, the mechanical position and the geometric deformation of the EPID may be calibrated in real time based on the pose calibration parameter, to eliminate the impact of the mechanical deformation of the support structure of the EPID and the impact of the non-uniformity of the EPID pixel responses on the analysis result of the measurement data of other QA objects. Therefore, the current measurement data corresponding to each QA object may be obtained accurately.

In the technical solution of this embodiment, the calibration parameter includes the pose calibration parameter corresponding to the EPID. By calibrating the portal image of the QA object based on the pose calibration parameter, the target portal image may be obtained. The current measurement data corresponding to each QA object are determined according to the image features of the target portal image. Such that, in the process of processing the portal images, the impact of the position change of the EPID on the measurement result may be eliminated, and the current measurement data corresponding to each QA object may be accurately obtained, thereby obtaining the accurate QA result corresponding to the QA object, and improving the QA accuracy of the radiotherapy device effectively.

In one of the embodiments, when the QA object includes an isocenter, the target portal images corresponding to the isocenter measurement include phantom images corresponding to the QA phantom at each of the preset gantry rotation angles, and square field images formed by a beam-limiting system at each of the gantry rotation angles, or include phantom images of the QA phantom in corresponding fields at each preset gantry rotation angle. The QA phantom includes multiple mark points.

The beam-limiting system includes a jaw and/or an MLC of the QA object.

Determining the current measurement data corresponding to each QA object according to the image features of the target portal image includes following steps. A projection matrix corresponding to each gantry rotation angle is determined according to mark-point spatial coordinates of the multiple mark points at initial positions, and according to the mark-point projection coordinates of multiple mark points in each phantom image. A straight-line trail of the radiation beam axis in a spatial coordinate system corresponding to the radiation beam axis at each gantry rotation angle is determined, according to the projection matrix corresponding to each gantry rotation angle, and the projection coordinates of the radiation field center corresponding to the radiation field center in the square field image under each gantry rotation angle. The radiation isocenter parameter corresponding to the isocenter is determined according to an intersection point of straight-line trails of radiation beam axes.

The mark-point spatial coordinates are the coordinates of the mark point in the spatial coordinate system corresponding to the QA phantom.

The target mark point is the mark point with the smallest diameter in the candidate mark point set.

The radiation isocenter parameter is configured to determine the current measurement data corresponding to the isocenter.

The mark-point projection coordinates are the projection pixel coordinates corresponding to the mark point in the phantom image.

The projection coordinates of the radiation field center are the projection pixel coordinates of the radiation field center in the square field image.

The above phantom images and the square field images are acquired by the EPID, when the QA phantom is moved into the radiation field range of the EPID based on a preset table position, and when the shape of the radiation field of the beam-limiting system is configured as a square field capable of covering the QA phantom.

The radiation field shape of the beam-limiting system may be configured as a square field of size 20 cm×20 cm to cover the QA phantom, or a square field of any other size, which is not limited herein.

Each preset gantry rotation angle may be the angle of the gantry when the gantry is in a different rotation position. The intervals between adjacent preset gantry rotation angles are equal. For example, the interval may be 45°, i.e., the preset gantry rotation angles are 45°, 90°, 135°, 180°, 225°, 270°, 315°, and 0°. The intervals between adjacent gantry rotation angles are not limited herein.

The phantom image acquired when the gantry rotation angle is 0° is the phantom image of the QA phantom at the initial position.

The spatial coordinate position of each of the mark points of the QA phantom relative to the position of the center of the QA phantom are known, i.e., the mark-point spatial coordinates of each mark point in the spatial coordinate system corresponding to the QA phantom are known.

In practical implementation, the QA phantom may be a BB phantom with a certain number (the number is greater than or equal to 12) of steel balls, for example, the BB (a steel ball) phantom with 18 steel balls (or other steel mark points).

In a specific implementation, when the QA object includes an isocenter, in the process of determining the current measurement data corresponding to each QA object based on the pose calibration parameter and the image features of the target portal image, the radiotherapy device may determine the mark-point projection coordinates of the projection positions of the mark points in each phantom image, and may determine the projection coordinates of the radiation field center corresponding to the projection position of the radiation field center in each square field image. In order to facilitate understanding of those skilled in the art, FIG. 5 shows phantom images and square field images acquired at gantry rotation angles (45°, 90°, 135°, 180°, 225°, 270°, 315°, and 0°) spaced at a certain angle. A circular mark point denotes a projection position of a mark point, and the crossline denotes the projection position of the radiation field center. The radiotherapy device may determine the projection matrix corresponding to each gantry rotation angle according to the mark-point spatial coordinates corresponding to the initial positions of the mark points, and the mark-point projection coordinates corresponding to the mark points in each phantom image.

For any gantry rotation angle, the relationship between the mark-point spatial coordinates corresponding to the mark point, the mark-point projection coordinates corresponding to the mark point, and the projection matrix is as follows:

$$[ \begin{matrix}λ {u}_{i} \\ λ {v}_{i} \\ λ\end{matrix} ] = P m a t * [ \begin{matrix}{x}_{i} \\ {y}_{i} \\ {z}_{i} \\ 1\end{matrix} ] = [ \begin{matrix}{p}_{11} & {p}_{12} & {p}_{13} & {u}_{0} \\ {p}_{21} & {p}_{22} & {P}_{23} & {v}_{0} \\ {p}_{31} & {p}_{32} & {p}_{33} & 1\end{matrix} ] [ \begin{matrix}{x}_{i} \\ {y}_{i} \\ {z}_{i} \\ 1\end{matrix} ]$$

Where xᵢ, yᵢ, and zᵢ denote the mark-point spatial coordinates corresponding to the i-th mark point respectively. uᵢ and vᵢ denote the mark-point projection coordinates corresponding to the i-th mark point respectively. u₀ and v₀ denote the projection coordinates of the radiation beam center corresponding to the projection position of the beam center respectively. λ denotes a proportion coefficient, and Pmat denotes the projection matrix.

In this way, the projection matrixes at different gantry rotation angles may be solved by the least square method. Then, the straight-line trail of the radiation beam axis in the spatial coordinate system corresponding to the radiation beam axis at each gantry rotation angle may be determined, according to the projection matrix corresponding to each gantry rotation angle and the projection coordinates of the radiation field center corresponding to the radiation field center in the square field image at each gantry rotation angle. Specifically, each gantry angle corresponds to a projection matrix, and then the radiation beam axis in the space may be reconstructed based on the projection coordinates (u₁, v₁) of the radiation field center corresponding to the radiation field center in the square field image at the gantry angle. The spatial points in the straight-line trail of the radiation beam axis corresponding to the radiation beam axis may be expressed as follows:

$$[ \begin{matrix}x \\ y \\ z\end{matrix} ] = {[ \begin{matrix}{p}_{11} & {p}_{12} & {p}_{13} \\ {p}_{21} & {p}_{22} & {p}_{23} \\ {p}_{31} & {p}_{32} & {p}_{33}\end{matrix} ]}^{- 1} [ \begin{matrix}λ {u}_{1} - {u}_{0} \\ λ {v}_{1} - {v}_{0} \\ λ - 1\end{matrix} ]$$

The coordinates (x, y, z) of the spatial points in the straight-line trail of the radiation beam axis may be determined by selecting different proportion coefficients k.

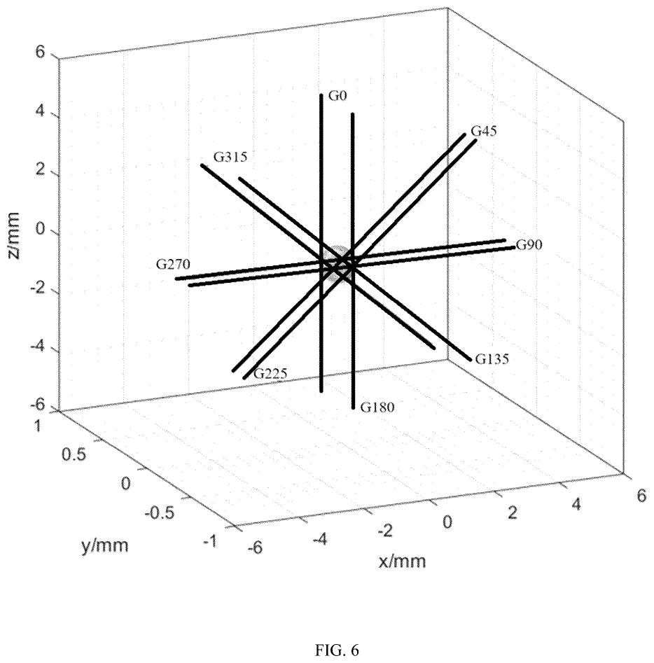

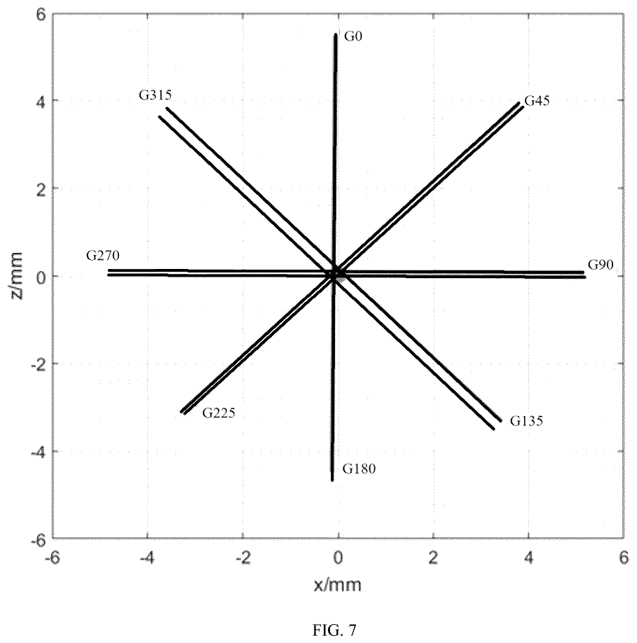

In order to facilitate the understanding of those skilled in the art, FIG. 6 shows straight-line trails of the radiation beam axes in the spatial coordinate system corresponding to the QA phantom at different gantry rotation angles, when a radiation beam axis rotates around the gantry, and FIG. 7 shows projections of the straight-line trails of the radiation beam axes shown in FIG. 6 on the XZ plane. Then the radiotherapy device, based on the spatial position relationship between the straight-line trails of the radiation beam axes may determine the intersection point of all straight-line trails of the radiation beam axes in the spatial coordinate system as the target mark point, and obtain the radiation isocenter size according to the diameter of the target mark point, to obtain the radiation isocenter parameter corresponding to the isocenter. Finally, the current measurement data corresponding to the isocenter may be determined based on the radiation isocenter parameter.

In addition, when the positioning laser for is aligned with QA phantom marker (at this time, the coordinate system with the phantom center as the origin is aligned with the spatial coordinate system), the mark-point spatial coordinates corresponding to the target mark point are used to reflect the deviation of laser from the isocenter.

In the technical solutions of this embodiment, when the QA object includes an isocenter, the target portal images corresponding to the isocenter measurement include the phantom image of the QA phantom at each preset gantry rotation angle and the square field image formed by the beam-limiting system at each preset gantry rotation angle. The QA phantom includes multiple mark points. The projection matrix corresponding to each gantry rotation angle may be accurately determined according to the mark-point spatial coordinates corresponding to the multiple mark points of the QA phantom and according to the mark-point projection coordinates corresponding to the multiple mark points in each phantom image. Therefore, according to the projection matrix corresponding to each gantry rotation angle and the projection coordinates of the radiation field center corresponding to the radiation field center in each square field image, the straight-line trails of the radiation beam axes in the spatial coordinate system at different gantry rotation angles may be accurately determined, to determine the target mark point, and then the isocenter size may be measured according to the diameter of the target mark point to obtain high-precision radiation isocenter parameters.

In one of the embodiments, when the QA object includes a table, the target portal images corresponding to the table include a first initial phantom image and first post-movement phantom images of the QA phantom.

The first initial phantom image is a phantom image of the QA phantom at the first gantry angle and at the initial position.

The first gantry angle may be a gantry rotation angle among the preset gantry rotation angles, for example, it may be 45°.

The first post-movement phantom images include a phantom image corresponding to the direction of each coordinate axis of the spatial coordinate system. Specifically, the first post-movement phantom images are phantom images of the QA phantom corresponding to different directions after the table is moved in the directions of coordinate axes of the spatial coordinate system, respectively, by taking the table position corresponding to the first initial phantom image as a starting position of the table.

More specifically, the first post-movement phantom images includes a phantom image of the QA phantom corresponding to an x-direction offset after the table is moved in the x-axis direction of the spatial coordinate system, a phantom image of the QA phantom corresponding to a y-direction offset after the table is moved in the y-axis direction of the spatial coordinate system, and a phantom image of the QA phantom corresponding to a z-direction offset after the table is moved in the z-axis direction of the spatial coordinate system. The first post-movement phantom images may further include the phantom image of the QA phantom corresponding to an ISO-angle offset after the table is rotated at a certain angle around the ISO axis (a rotation axis).

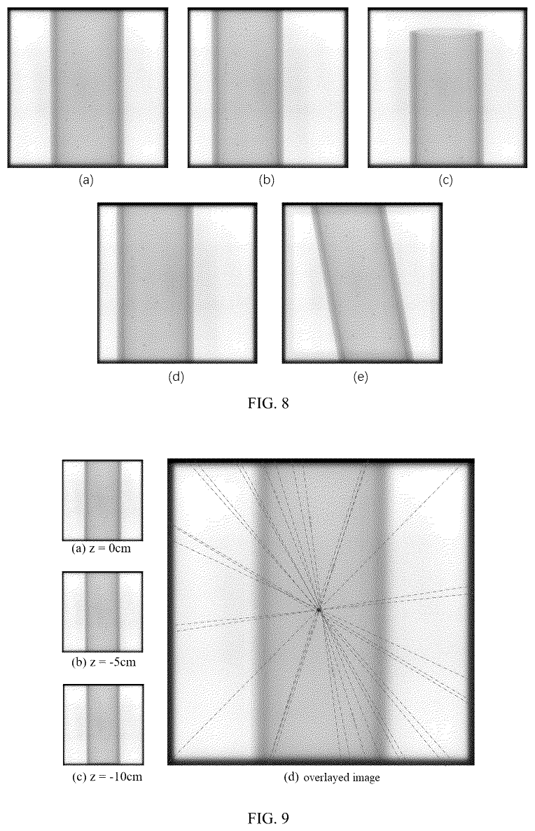

In order to facilitate understanding of those skilled in the art, FIG. 8(a) to FIG. 8(e) respectively show a first initial phantom image, a phantom image corresponding to an x-direction offset, a phantom image corresponding to a y-direction offset, a phantom image corresponding to a z-direction offset, and a phantom image corresponding to an ISO-angle offset.

The determining the current measurement data corresponding to each QA object according to the image features of the target portal image, includes following steps: determining post-movement mark-point projection coordinates of multiple mark points on coordinate axes according to each first post-movement phantom image; and determining actual movement parameters of the table in the coordinate axes respectively corresponding to the gantry angle and the post-movement mark-point projection coordinates, which specifically includes: determining the post-movement mark-point spatial coordinates of the multiple mark points in coordinate axes according to a projection matrix corresponding to a first gantry angle and the post-movement mark-point projection coordinates; and determining actual movement parameters of the table in coordinate axes respectively according to the post-movement mark-point spatial coordinates of the multiple mark points in each coordinate axis, and according to a mapping relationship among the mark-point spatial coordinates of the multiple mark points at the first gantry angle. The actual movement parameters are used to determine the current measurement data corresponding to the table.

In a specific implementation, when the QA object is the table, in the process of determining the current measurement data corresponding to each QA object based on the image features of the target portal image, in order to improve the measurement accuracy, the radiotherapy device may establish a relationship by using changes of the mark-point spatial coordinates and changes of the mark-point projection coordinates, thereby calculating the actual movement parameter in each coordinate axis of the table.

Specifically, the radiotherapy device may determine the post-movement mark-point projection coordinates of the mark points on different coordinate axes based on each first post-movement phantom image. Then, the radiotherapy device may determine the post-movement mark-point spatial coordinates of the mark points in each coordinate axis, according to a projection matrix corresponding to the first gantry angle and the post-movement mark-point projection coordinates. Finally, the radiotherapy device may determine a translation parameter and a rotation parameter of the table in each coordinate axis as the actual movement parameters of the table in each coordinate axis, according to the post-movement mark-point spatial coordinates corresponding to each mark point under each coordinate axis, and according to the mapping relationship among the mark-point spatial coordinates under the first gantry angle, thereby determining the current measurement data corresponding to the table according to the actual movement parameters of the table in each coordinate axis.

In a practical application, the changes of the mark-point spatial coordinates of the i-th mark point due to the movement of the table may be as follows:

$$[ \begin{matrix}{x}_{i} \\ {y}_{i} \\ {z}_{i}\end{matrix} ] = {R}_{z} {R}_{y} {R}_{x} [ \begin{matrix}{x}_{0} \\ {y}_{0} \\ {z}_{0}\end{matrix} ] + [ \begin{matrix}{T}_{x} \\ {T}_{y} \\ {T}_{z}\end{matrix} ]$$

where (x₀, y₀, z₀) denote the mark-point spatial coordinates of the mark point of the QA phantom when the first initial phantom image of the QA phantom is acquired at the first gantry angle. Rₓ, R_{y}, and R_{z} denote the rotation matrixes of the table around each coordinate axes; Tₓ, T_{y}, and T_{z} denote the translation displacements of the table along each coordinate axis; and (xᵢ, yᵢ, zᵢ) are the post-movement mark-point spatial coordinates of the i-th mark point after the movement of the table.

Rₓ, R_{y}, and R_{z} may be as follows.

$$\begin{matrix}{R}_{x} = [ \begin{matrix}1 & 0 & 0 \\ 0 & cos ( roll ) & sin ( roll ) \\ 0 & - s in ( roll ) & cos ( roll )\end{matrix} ] \\ {R}_{y} = [ \begin{matrix}cos ( pitch ) & 0 & - s in ( pitch ) \\ 0 & 1 & 0 \\ sin ( pitch ) & 0 & cos ( pitch )\end{matrix} ] \\ {R}_{z} = [ \begin{matrix}cos ( yaw ) & sin ( yaw ) & 0 \\ - s in ( yaw ) & cos ( yaw ) & 0 \\ 0 & 0 & 1\end{matrix} ]\end{matrix}$$

where roll denotes the rotation angle of the table around the X-axis, pitch denotes the rotation angle of the table around the Y-axis, and yaw denotes the rotation angle of the table around the Z-axis.

The change of the mark-point projection coordinates of the i-th mark point due to the movement of the table may be as follows:

$$[ \begin{matrix}λ {u}_{i} \\ λ {v}_{i} \\ λ\end{matrix} ] = P m a {t}_{0} * [ \begin{matrix}{x}_{i} \\ {y}_{i} \\ {z}_{i} \\ 1\end{matrix} ]$$

where (uᵢ, vᵢ) denote the post-movement mark-point projection coordinates of the i-th mark point after the movement of the table, and Pmat₀ denotes a projection matrix corresponding to the first angle. Multiple equation systems may be established for multiple mark points and solved by the least square method. When the table moves along a coordinate axis, the translation displacement of the table along the coordinate axis and the rotation amount around the coordinate axis may be calculated according to the equations above, so that the translation parameters and rotation parameters of the table corresponding to each coordinate axis may be obtained.

In the technical solution of this embodiment, when the QA object is the table, the target portal images corresponding to the table include the first initial phantom image and the first post-movement phantom images. The first post-movement phantom images include a phantom image corresponding to the direction of each coordinate axis of the spatial coordinate system. The QA phantom includes multiple mark points. The post-movement mark-point projection coordinates of multiple mark points on different coordinate axes are determined according to each first post-movement phantom image. The post-movement mark-point spatial coordinates of the multiple mark points in coordinate axes are determined according to the projection matrix corresponding to the first gantry rotation angle and the post-movement mark-point projection coordinates. The actual movement parameters of the table in coordinate axes are determined respectively according to the post-movement mark-point spatial coordinates of the multiple mark points in each coordinate axis, thereby establishing a relationship based on the changes of the mark-point spatial coordinates and changes of the mark-point projection coordinates, accurately calculating the actual movement parameter in each coordinate axis of the table, effectively improving the accuracy of the current measurement data corresponding to the table, and obtaining the accurate QA result of the table.

In one of the embodiments, when the QA object includes a gantry, the target portal image of the gantry includes a second initial phantom image and second post-movement phantom images of the QA phantom.

The second initial phantom image is a phantom image at the second gantry rotation angle.

The second gantry rotation angle may be a certain gantry rotation angle among the preset gantry rotation angles, for example, it may be 0°.

The second post-movement phantom images include phantom images at different heights of the table. Specifically, the second post-movement phantom image is a phantom image of the QA phantom after the table is moved along the height direction (Z-axis direction) for a preset distance by taking the table position corresponding to the first initial phantom image as a starting position of the table.

More specifically, the second initial phantom image may be the phantom image of the QA phantom at a certain height of the table (for example, Z=0 cm) at the second gantry rotation angle. The second post-movement phantom image may include phantom images of the QA phantom obtained respectively after the table is lowered along the Z-axis direction by a first preset distance (for example, the first preset distance is 5 cm, i.e., Z=−5 cm) or by a second preset distance (for example, the second preset distance is 10 cm, i.e., Z=−10 cm) based on a reference of the table position of the second initial phantom image.

In order to facilitate understanding of those skilled in the art, FIG. 9(a) shows a second initial phantom image, and FIG. 9(b) and FIG. 9(c) show a second post-movement phantom image respectively.

The determining the current measurement data corresponding to the QA object according to the image features of the target portal image, includes following steps. For the same mark point, target mark-point projection coordinates in the second initial phantom image and in each of the second post-movement phantom images are determined to obtain the current measurement data corresponding to the gantry. Specifically, in each of the second post-movement phantom images and the second initial phantom image, a projection-position scaling path corresponding to the mark point is obtained according to the target mark-point projection coordinates corresponding to the same mark point; and an intersection point of the projection-position scaling paths corresponding to all mark points is determined, to obtain the current measurement data corresponding to the gantry.

The target mark-point projection coordinates are the projection pixel coordinates corresponding to the same mark point in the second initial phantom image and in each of the second post-movement phantom images.

In a specific implementation, when the QA object further includes the gantry, in the process of determining the current measurement data corresponding to each QA object based on the image features of the target portal image, the radiotherapy device may determine the target mark-point projection coordinates in the second initial phantom image and in each of the second post-movement phantom images. Therefore, the radiotherapy device may superimpose each of the second post-movement phantom images and the second initial phantom image to obtain the superimposed phantom image, and the radiotherapy device may determine the connection line connecting all target mark-point projection coordinates in the superimposed phantom image, which corresponds to the same mark point, and use the connection line as the projection-position scaling path corresponding to the mark point, thus obtaining the projection-position scaling paths corresponding to all mark points in the superimposed phantom image. Then, according to the projection-position scaling paths corresponding to all mark points, the radiotherapy device may determine a point P in the image, where the projection pixel coordinate position remains unchanged during the lifting and lowering process of the table. The point P is the intersection point of the projection-position scaling paths corresponding to all mark points. This intersection point is related to the gantry rotation angle. The current measurement data corresponding to the gantry may be determined according to the intersection point.

In order to facilitate the understanding of those skilled in the art, FIG. 9(d) shows an intersection point of the projection-position scaling paths corresponding to all mark points in a superimposed phantom image. Each dotted line in FIG. 9(d) denotes a projection-position scaling path corresponding to a mark point.

Specifically, in the process of determining the intersection point of the projection-position scaling paths corresponding to all mark points, and determining the current measurement data corresponding to the gantry based on the intersection point, the radiotherapy device may determine the projection pixel coordinates of the intersection point in the superimposed phantom image as the intersection-point projection coordinates. Then, when performing a QA on the isocenter, the radiotherapy device may use the coordinates of the target mark point in the spatial coordinate system as the isocenter spatial coordinates of the isocenter in the spatial coordinate system, and determine the projection pixel coordinates P0 of the isocenter in the superimposed phantom image (i.e., in the plate detector of the EPID) as the isocenter projection coordinates according to the projection matrix corresponding to the second gantry rotation angle at which the second initial phantom image is acquired, and according to the isocenter spatial coordinates. In this way, the radiotherapy device may determine the current measurement data corresponding to the gantry based on a target distance between the isocenter projection coordinates and the intersection-point projection coordinates.

Specifically, in the process of determining the current measurement data corresponding to the gantry according to the target distance between the isocenter projection coordinates and the intersection-point projection coordinates, the radiotherapy device may determine the angle Gangle of the gantry as the current measurement data corresponding to the gantry, based on the target distance PP0 between the isocenter projection coordinates and the intersection-point projection coordinates and based on the current measured source-to-imager distance (SID). Gangle may be as follows:

Gangle=arctan(PP0/SID)

In this way, when the second gantry rotation angle is 0°, the zero-position angle deviation of the gantry may be evaluated based on Gangle, to obtain the QA result of the gantry.

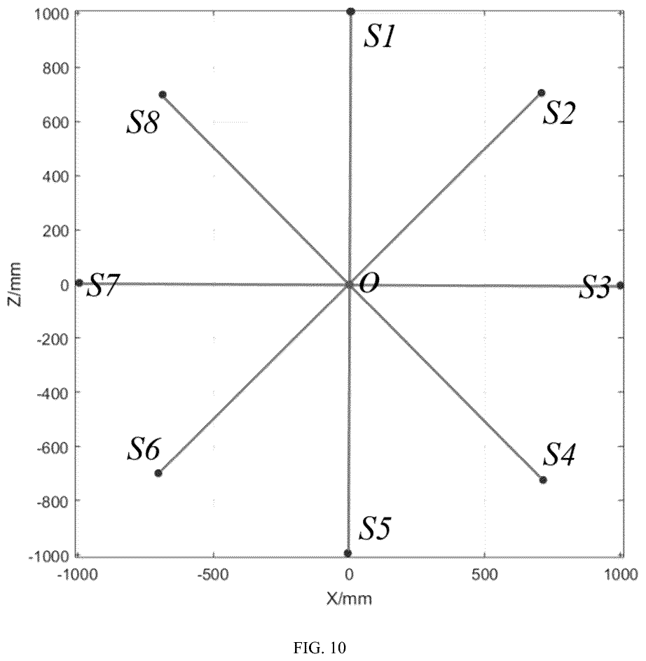

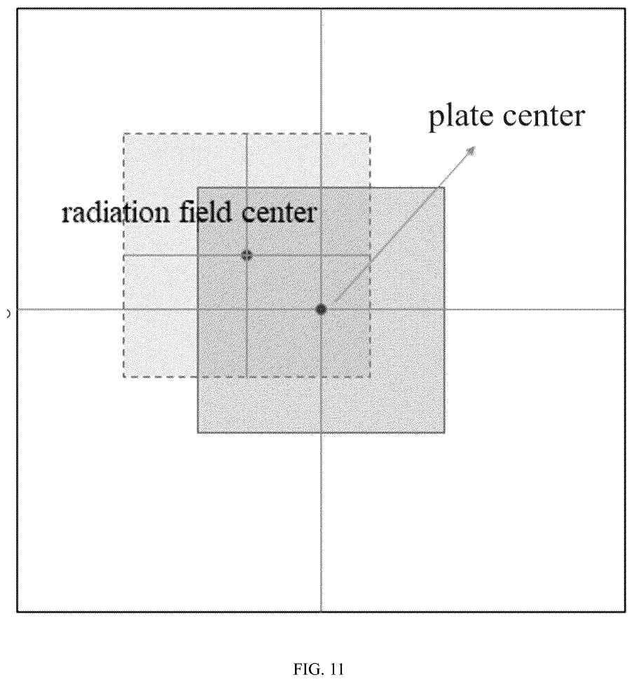

In addition, when performing the QA on the isocenter, corresponding to λ=0, all straight-line trails of the radiation beam axes passing through the light source of the radiotherapy device will converge to one point, namely, the light source point. In order to facilitate the understanding of those skilled in the art, FIG. 10 shows a change of position of the light source points (S1, S2, . . . , and S8) under different preset gantry rotation angles, where point O is the isocenter. Referring to FIG. 10, during the gantry rotation, changes of the coordinates of the light source point in the spatial coordinate system, namely the changes of the spatial coordinates of the light source point, may be determined. At different gantry rotation angles, after the coordinates of the light source point in the spatial coordinate system, i.e., the spatial coordinates of the light source point, are determined, the current gantry rotation angle may be determined based on the isocenter spatial coordinates. For example, an angle between the positions of different light source points relative to the isocenter may be calculated in a manner of a vector. The angle is the current rotation angle of the gantry. By comparing the current gantry rotation angle with the preset gantry rotation angle, the rotation accuracy of the gantry may be determined. As shown in FIG. 10, S₁ denotes a specific light source point position, and the preset gantry rotation angle of the light source point position S₁ is θ₁. S₂ denotes another specific light source point position. The preset gantry rotation angle corresponding to the light source point position S₂ is θ₂. By calculating the current rotation angle ∠S₁OS₂ of the gantry, and comparing current rotation angle with the preset gantry rotation angle (θ₂−θ_{1,}), the rotation accuracy of the gantry may be obtained.

In this way, the QA result of the gantry may be obtained based on the rotation accuracy and the zero-position angle deviation of the gantry.

In the technical solution of this embodiment, when the QA object further includes the gantry, the target portal images of the gantry include a second initial phantom image and second post-movement phantom images. The second post-movement phantom images include phantom images at different heights of the table. By superimposing the second post-movement phantom images and the second initial phantom image, and in the obtained superimposed phantom image, the intersection point of the projection-position scaling paths corresponding to all mark points is determined, so that one point in the image, whose projection pixel coordinates remain unchanged during the lifting and lowering process of the table, may be determined. This point is related to the gantry angle, therefore, the angle deviation of the gantry may be accurately determined based on the intersection-point projection coordinates of this point and the isocenter projection coordinates of the isocenter, to obtain the accurate QA result of the gantry.

In one of the embodiments, when the QA object includes a beam component, the target portal image of the beam component includes a preset number of square field images. The determining the current measurement data corresponding to each QA object according to the image features of the target portal image includes following steps. The square field images are superimposed according to a position of radiation field center and a position of an image center to obtain a superimposed square field image. The superimposed square field image is translated to obtain a translated square field image. Specifically, the radiation field center of the translated square field image is aligned with an image center. The translated square field image is adjusted according to the image scaling coefficient, to obtain a target square field image. The target square field image is used as the current measurement data corresponding to the beam component.

The image center is the center of the superimposed square field image, namely a plate center of the plate detector of the EPID.

In a specific implementation, when the QA object includes a beam component, an MLC and/or a jaw may be used to form a radiation field that may cover the imaging area of the plate of the EPID as much as possible. In order to remove the influence of residual signals of the EPID, the EPID may acquire a certain number (such as 5 or other numbers) of dark field images under unexposed conditions. In the case that the radiation field shape of the MLC and/or jaw is configured as a square field of a certain size (for example, 25 cm×25 cm or a square field of any other size), in order to allow the beam to be in a stable state, each image may be given a dose of 1MU, and a certain number (such as 10 or any other number) of square field images may be continuously acquired and used as the target portal image of the beam component.

Each dose of 1MU is limited by the maximum close (saturation dose) that the plate detector of the EPID may accept. If the saturation dose of the plate detector is very high, the number of acquired square field images may be reduced. In practical application, the number of dark field images and the number of square field images may have other number.

In this way, when the QA object includes a beam component, in the process of determining the current measurement data corresponding to each QA object according to the image features of the target portal image, the radiotherapy device may superimpose the preset number of square field images to obtain the superimposed image. The radiotherapy device may perform a bad pixel calibration, a plate detector gain calibration (removing the inconsistency of each pixel response), and a mean filtering, etc., for the superimposed image to obtain the superimposed square field image. Then, the radiotherapy device may determine the position of the radiation field center and the image center of the superimposed square field image, and translate the superimposed square field image based on the position of the radiation field center and the position of the image center (a plate center), to translate the position of the radiation field center to the position of the image center (the plate center), so that the radiation field center and the image center (the plate center) are aligned to obtain the translated square field image, thus avoiding the problem that large image differences are caused when the radiation field illuminates different positions on the plate detector due to the poor repeatability of the plate detector of the EPID, thereby eliminating the influence of the mechanical repeatability of the EPID. In order to facilitate understanding of those skilled in the art, FIG. 11 is a schematic view of shifting the position of the radiation field center to the position of the image center (the plate center). The offset CS of the radiation field center in the superimposed square field image may characterize the position difference of the radiation field center of the beam component relative to a rotation center of the collimator.

Then, the radiotherapy device may perform a scaling operation on the translated square field image according to the second calibration parameter corresponding to the EPID, to obtain the scaled and translated square field image, and then may adjust the pixel values of the scaled and translated square field image to obtain the target square field image. The pixel values of the image acquired by the EPID are inversely proportional to the square of SID. The target square field image will be used as the current measurement data corresponding to the beam component.

In this way, when the QA object is the beam component, in the process of determining a QA result of the QA object of the radiotherapy device according to the current measured each QA object, the radiotherapy device may determine a ratio image of the target square field image to a reference square field image, based on the target square field image and the reference square field image corresponding to the beam component. The reference square field image corresponding to the beam component is a reference status image corresponding to the beam component, which is acquired after the radiotherapy device is debugged and accepted. The method of obtaining the reference square field image is the same as the method of obtaining the target square field image, and will not be described repeatedly herein.

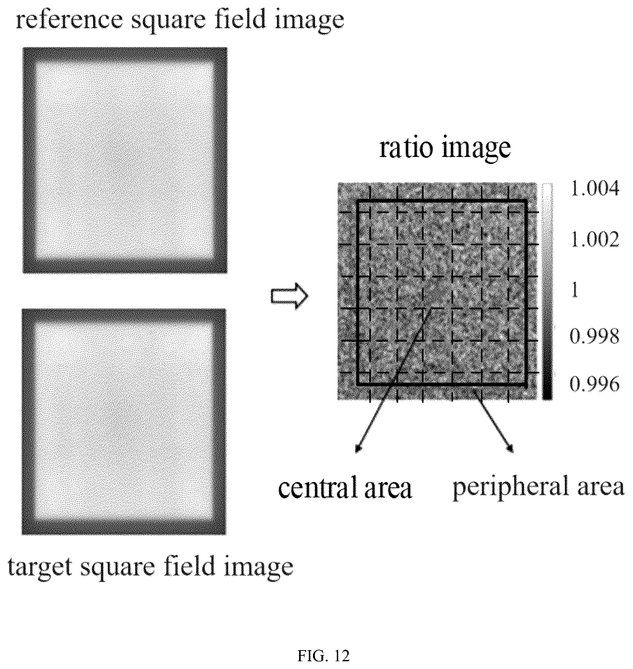

Then, the radiotherapy device may divide the ratio image into regions, and determine a central area and a peripheral area in the divided ratio image. The ratio of the area of the peripheral area to the area of the ratio image satisfies a preset ratio condition, and the center position of the central area is the same as the center position of the ratio image. In practical application, in order to reduce the impact of the radiation field boundary and single-pixel noise on the QA result of the beam component, the ratio image may be divided into N×N areas, where N is greater than 3, for example, N=7, and a certain proportion (for example, 80%) of the radiation field of the regionally divided ratio image is used as the peripheral area. In order to facilitate the understanding of those skilled in the art, FIG. 12 shows a reference square field image, a target square field image, and a ratio image. The division of the center area and the peripheral area is shown in FIG. 12.

Then, the radiotherapy device may determine a dose output change parameter corresponding to the beam component based on the mean pixel value difference of the pixels in the central area. Based on the maximum difference of differences of grey levels between a pixel block in the peripheral area and a pixel block in the central area, the radiotherapy device may determine a change parameter of output beam uniformity corresponding to the beam component. The change parameter of output beam uniformity characterizes a change of beam energy. Specifically, the above parameters may be as follows:

$$\begin{matrix}OC = ( {\sum }_{( x , y ∈ CROI )} R I ( x , y ) / {N}_{CROI} - 1 ) * 100 \% \\ \{ \begin{matrix}{ECDiff}_{i} = {\sum }_{x , y , ∈ {EROI}_{i}} \frac{RI ( x , y )}{{N}_{{EROI}_{i}}} - {\sum }_{x , y ∈ CROI} \frac{RI ( x , y )}{{N}_{CROI}} \\ UC = max ( \vert {ECDiff}_{1} \vert , \vert {ECDiff}_{2} \vert , …… \vert {ECDiff}_{i} \vert )\end{matrix}\end{matrix}$$

where OC denotes the dose output change parameter; RI denotes the ratio image; CROI denotes the central area; (x, y) denotes the pixel coordinates; N_{CROI} denotes the number of pixels in the central area; ECDiffᵢ denotes the difference of grey level change between the pixel block in the peripheral area and a pixel block in the central area; EROI denotes the peripheral area; N_{EROIi}, denotes the number of pixels in a pixel block in the peripheral area; and UC denotes the change parameter of output beam uniformity.

In this way, the radiotherapy device may determine the QA result of the beam component based on the dose output change parameter and the change parameter of output beam uniformity, to monitor the stability of the beam component.

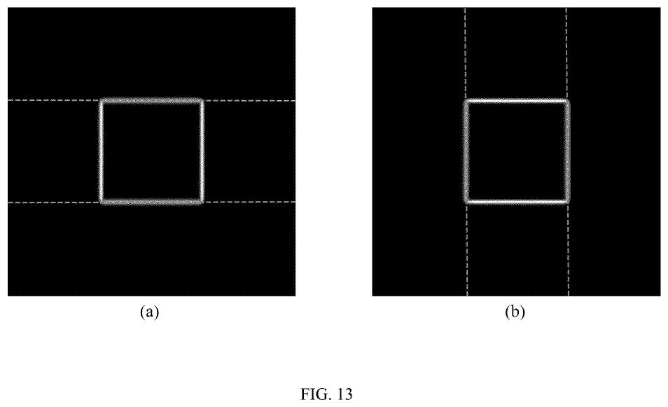

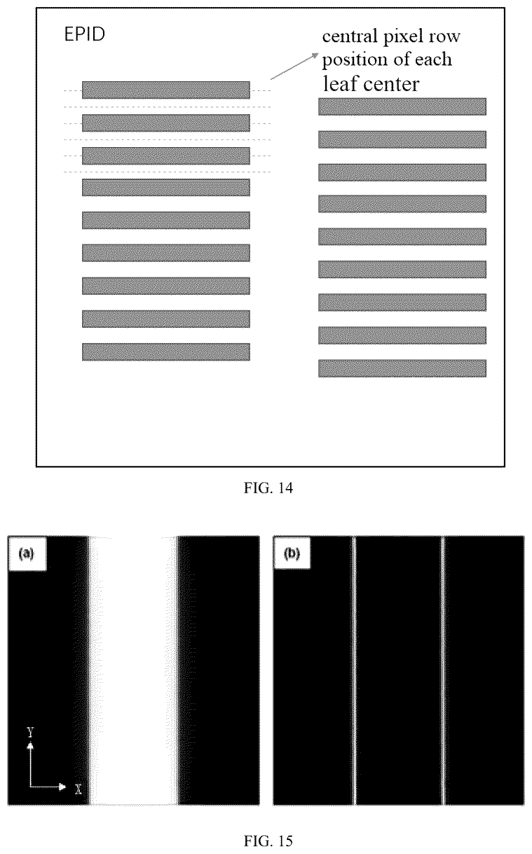

In one of the embodiments, when the QA object includes a collimator, the target portal image corresponding to the collimator includes a correction square field rotation image (serving as the first correction square field rotation image) corresponding to the collimator angle of 0°, and a correction square field rotation image (serving as the second correction square field rotation image) corresponding to the collimator angle of 90°. When the QA is performed on the collimator, a zero-position change and rotation accuracy of the collimator are mainly monitored. In this process, the first correction square field rotation image and the second correction square field rotation image in the correction square field image are multiplexed. In this way, in the process of determining the current measurement data corresponding to the QA object according to the image features of the target portal image, the radiotherapy device may perform a gradient transformation on the first correction square field rotation image and the second correction square field rotation image, to obtain the first correction square field gradient image and the second correction square field gradient image. Then the radiotherapy device may fit the boundary of the jaw in the first correction square field gradient image, to obtain the first fit boundary line. The radiotherapy device may fit the boundary of the jaw in the second correction square field gradient image, to obtain the second fit boundary line. In order to facilitate understanding of those skilled in the art, FIG. 13(a) shows first fit boundary lines in the first correction square field gradient image, and FIG. 13(b) shows second fit boundary lines in the second correction square field gradient image, and the dotted lines in the figures denote the fit boundary lines. Then the radiotherapy device may determine a first boundary measurement inclination angle (i.e., a measurement inclination angle of 0° corresponding to a collimator angle of 0°) corresponding to the first fit boundary line, and determine a second boundary measurement inclination angle (i.e., a measurement inclination angle of 90° corresponding to a collimator angle of 90°) corresponding to the second fit boundary line. The current measurement data corresponding to the collimator are obtained according to the first boundary measurement inclination angle and the second boundary measurement inclination angle.

In this way, when the QA object is collimator, in the process of determining the QA result corresponding to the radiotherapy device according to the current measurement data corresponding to each QA object, the radiotherapy device may compare the measurement inclination angle of 0° with the reference inclination angles corresponding to the collimator in the reference state, to obtain the zero-position change of the collimator, which is an average difference of differences between two first boundary measured inclination angles and two reference inclination angles. The radiotherapy device may obtain the measured rotation angle of the collimator according to the difference between the measured inclination angle of 0° and the measured inclination angle of 90°. The radiotherapy device may compare the measured rotation angle with the target rotation angle corresponding to the collimator in the reference state, to obtain the rotation accuracy of the collimator, which is an average difference between the two rotation angles. Therefore, the radiotherapy device can obtain the QA result of the collimator based on the zero-position change of the collimator and the rotation accuracy of the collimator.

In one of the embodiments, when the QA object includes an MLC. In the process of performing the QA on the MLC, the target portal image of the MLC includes a collimator portal image acquired after the shape of the radiation field of the MLC is set to a specific size. In the process of determining the current measurement data corresponding to each QA object based on the image features of the target portal image, the radiotherapy device may determine the longitudinal positions (in Y-direction) of the leaves of the MLC in the collimator portal image according to the position of each central pixel row where each leaf center is located, and establish a radiation field coordinate system in the collimator portal image. Where, the position of each central pixel row is included in the second calibration parameter of the EPID. In order to facilitate the understanding of those skilled in the art, FIG. 14 is a schematic view showing positions of central pixel rows where centers of leaves are located respectively in a comb-shaped beam image. FIG. 15(a) shows a radiation field coordinate system in a collimator portal image. Then, the radiotherapy device may perform a transverse (X-direction) gradient transformation on the collimator portal image. Since the positions of central pixel rows where centers of leaves are located respectively may be configured to distinguish multiple collimators, the radiotherapy device, based on the position of each central pixel row, may determine the position of the pixel, which has the largest gray gradient change corresponding to each leaf, and which is in the collimator portal image on which the transverse gradient transformation is performed, and use the position of the pixel as the pixel position to be converted corresponding to each leaf. In order to improve the positioning accuracy, the pixel position may be subdivided by the method of cubic spline interpolation or the method of Gaussian fitting. In order to facilitate understanding of those skilled in the art, FIG. 15(b) shows a collimator portal image on which a transverse gradient transformation is performed.

Then the radiotherapy device may convert the pixel position to be converted into the corresponding radiation field position in the radiation field coordinate system, to obtain the current measurement position of each leaf, and use the current measurement position of each leaf as the current measurement data corresponding to the MLC. In this way, when the QA object is the MLC, in the process of determining the QA result corresponding to the radiotherapy device according to the current measurement data corresponding to each QA object, the radiotherapy device may compare the current measurement position of each leaf with the reference position of each leaf in the MLC, to obtain the leaf offset. The leaf offset includes the average leaf offset of the leaves bank1/bank2 (left leaf group and right leaf group of the MLC), and the maximum leaf offset (bank1 and bank2 are determined separately). The reference position of each leaf is determined according to the collimator reference portal image of the MLC. The collimator reference portal image is a reference state image of the MLC acquired after the radiotherapy device is debugged and accepted. The method of obtaining the reference position is the same as the method of obtaining the current measurement position of the leaf in the process of the QA, and will not be described repeated hereinafter.

In addition, in the process of performing the QA on the MLC, the radiotherapy device may also determine the size of the radiation field of the MLC and other indicators such as the offset of the radiation field center of the MLC relative to the rotation center of the collimator, according to the size of the radiation field image of the MLC and the position of the radiation field center in the collimator portal image, so that the QA result of the MLC may be determined based on above indicators and the leaf offset, to monitor the change of the state of the MLC.

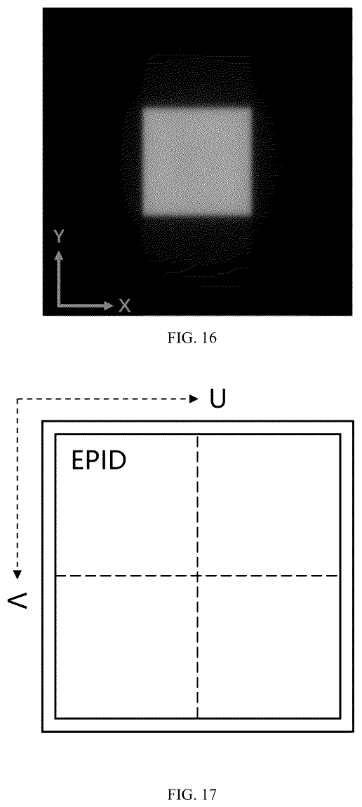

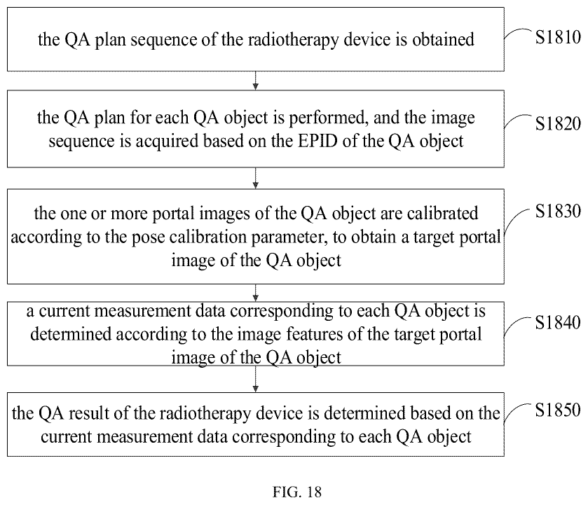

In one of the embodiments, when the QA object includes a jaw, in the process of performing the QA on the jaw, the target portal image of the jaw includes the jaw beam image acquired after the shape of the radiation field of the jaw is configured to a set size. In the process of determining the current measurement data corresponding to each QA object according to the image features of the target portal image, the radiotherapy device may establish a radiation field coordinate system in the jaw beam image. In order to facilitate understanding of those skilled in the art, FIG. 16 shows a radiation field coordinate system in a jaw beam image. Then the radiotherapy device may perform longitudinal analysis on the jaw beam image. Specifically, the radiotherapy device may perform a longitudinal (Y-direction) gradient transformation on the jaw beam image, and determine the jaw pixel position, specifically, in the jaw beam image on which the longitudinal gradient transformation is performed, to obtain a longitudinal jaw measurement position. Specifically, the boundary positioning accuracy may be improved through interpolation, and the jaw pixel position may be converted into the corresponding radiation field position in the radiation field coordinate system to obtain the longitudinal jaw measurement position. In addition, the radiotherapy device may also perform horizontal analysis on the jaw beam image, so that the horizontal jaw measurement position may be obtained. The principle of the method of horizontal analysis is the same as that of the method of longitudinal analysis for the MLC, which will not be described in detail hereinafter. In this way, the radiotherapy device may use the longitudinal jaw measurement position and the transverse jaw measurement position as the current measurement data corresponding to the jaw.

In this way, when the QA object is the jaw, in the process of determining the QA result corresponding to the radiotherapy device based on the current measurement data corresponding to each QA object, the radiotherapy device may compare the longitudinal jaw measurement position and the transverse jaw measurement position with the reference positions of the jaw respectively, which include a horizontal reference position and a longitudinal reference position, to obtain each jaw offset. The reference positions of the jaw are determined according to the jaw reference portal image of the jaw, and the jaw reference portal image is the reference state image of the jaw, which is acquired after the radiotherapy device is debugged and accepted. The method of obtaining the reference positions is the same as the method of obtaining the longitudinal jaw measurement position and the transverse jaw measurement position in the process of the QA, and will not be described again hereinafter.

In this way, in the process of performing the QA on the jaw, the radiotherapy device may also determine the X/Y-direction offset of the radiation field center of the jaw in relative to the collimator rotation center, according to the position of the radiation field center in the jaw beam image. The radiotherapy device may determine the QA result of the jaw based on the X/Y-direction offset and each jaw offset, to monitor the change of the state of the jaw.

In one of the embodiments, when the EPID is used in image-guided radiation therapy (IGRT), the change of the position of EPID will directly affect the results of IGRT and the quality of reconstructed images, so the position status of the EPID needs to be monitored. When the QA object is the plate detector of the EPID, in the process of determining the current measurement data corresponding to each QA object according to the image features of the target portal image, the radiotherapy device may determine the current measurement position of the collimator rotation center in the plate coordinate system, by using the corresponding position of the collimator rotation center on the plate detector of the EPID, which is included in the third calibration parameter of the EPID, as a reference point. The plate coordinate system is a U/V coordinate system, namely a pixel coordinate system, corresponding to the plate detector. In order to facilitate understanding of those skilled in the art, FIG. 17 shows a U/V coordinate system corresponding to the plate detector.

In addition, the radiotherapy device may also determine the leaf lateral width of the MLC based on a correction comb-shaped beam image, and determine the distance from the plate detector to an X-ray source of the radiotherapy device based on the leaf lateral width as a current measured SID. Then, the radiotherapy device may obtain the current measurement data corresponding to the plate detector based on the current measurement position of the collimator rotation center in the plate coordinate system and the current measured SID.

In this way, when the QA object is the plate detector of the EPID, in the process of determining the QA result of the radiotherapy device based on the current measurement data corresponding to each QA object, the radiotherapy device may compare the current measurement position of the collimator rotation center in the plate coordinate system with the reference position of the collimator rotation center in the plate coordinate system, to obtain the U/V-direction offset of the plate detector, and compare the current measured SID with the reference SID, to obtain the SID offset of the plate detector. Thus, the radiotherapy device may determine the QA result of the plate detector based on the U/V-direction offset of the plate detector and the SID offset of the plate detector, thereby monitoring the change of the mechanical state of the EPID.

The reference position of the collimator rotation center in the plate coordinate system and the reference SID are determined based on the correction radiation field reference image. The correction radiation field reference image is the correction image acquired after the radiotherapy device is debugged and accepted. The method of obtaining the reference position and reference SID is the same as the above method of obtaining the current measurement position of the collimator rotation center in the plate coordinate system and the current measured SID, and will not be repeated hereinafter.

In another embodiment, as shown in FIG. 18, a QA method for a radiotherapy device is provided. The method is explained by taking the method applied to the radiotherapy device as an example, and includes the following steps S1810 to S1850.

In step S1810, the QA plan sequence of the radiotherapy device is obtained.

In step S1820, the QA plan for each QA object is performed, and the image sequence is acquired based on the EPID of the QA object.

In step S1830, the one or more portal images of the QA object are calibrated according to the pose calibration parameter, to obtain a target portal image of the QA object.

In step S1840, a current measurement data corresponding to each QA object is determined according to the image features of the target portal image.

In step S1850, the QA result of the radiotherapy device is determined based on the current measurement data corresponding to each QA object.

It should be noted that for the specific limitations of the above steps, please refer to the specific limitations of the QA method for the radiotherapy device described above.

In one of the embodiments, the present disclosure provides another QA method for a radiotherapy device, which is applied to the radiotherapy device. The QA method for the radiotherapy device includes following steps. The calibration parameter corresponding to the one or more correction images is determined based on the one or more correction images acquired by the EPID for correcting the EPID. The target portal image of a QA object is obtained based on the calibration parameter and the one or more portal images of the QA object acquired by the EPID. The QA result of the QA object of the radiotherapy device is determined according to the target portal image.

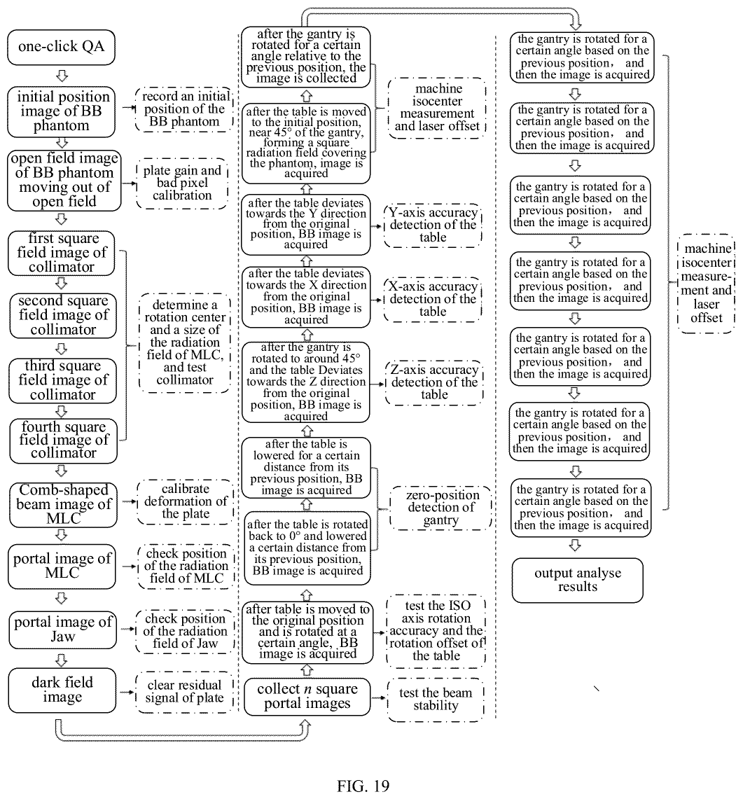

In one of the embodiments, as shown in FIG. 19, another QA method for a radiotherapy device is shown, and the method is applied to the radiotherapy device. The EPID may be configured to acquire an initial position image of the BB phantom in a preset initial position, to record the initial position of the BB phantom. Then, the BB phantom is moved out of the radiation field range of the EPID, and a correction open filed image is acquired and used for a bad pixel calibration and a plate gain calibration (equivalent to response calibration). The correction square field rotation images each corresponding to collimator angles spaced by a certain angle are acquired, and are configured to determine the collimator rotation center, the radiation field size of the MLC, and a collimator detection. The intervals between any adjacent collimator angles are equal, for example, the interval may be 90°, for example, the collimator angles are 180°, 90°, 0°, and 270° respectively. The intervals between any adjacent collimator angles are not specifically limited. The correction comb-shaped beam image (equivalent to the comb-shaped beam image of the MLC shown in FIG. 19) is acquired and configured to calibrate the deformation of the plate detector of the EPID. The portal images including the MLC, the jaw, the beam component, the table, the gantry, the isocenter measurement, and the laser offset, are acquired in sequence by following the arrows shown in FIG. 19, to obtain the QA result corresponding to each QA object of the radiotherapy device.

Specifically, the MLC portal image (collimator portal image) may be acquired for the MLC, and the MLC radiation field position may be checked. Then the jaw beam image is acquired for the jaw, and the jaw radiation field position is checked. Then a certain number of dark field images are acquired to remove the residual signal of the panel. Then a certain number of square field images are acquired for the beam component to test the beam stability. Then the table is moved so that the QA phantom is at an initial position (the gantry rotation angle is 0° in this case), and after the table is rotated for a certain angle along the ISO axis, the ISO-angle offset phantom image of the QA phantom is acquired to test the ISO axis rotation accuracy of the table by the rotation offset. The table is rotated back to the original position (i.e., the position of the table when the QA phantom is in the initial position), and the table is lowered from the original position for a certain distance along the height direction to acquire the phantom image of the QA phantom. The table is lowered for a certain distance again from the previously lowered position along the height direction, to acquire the phantom image of the QA phantom for gantry zero detection. Then the gantry rotation angle may be adjusted to be 45°, the table is moved to the original position and deviates towards the Z direction from the original position, to acquire the z-direction offset phantom image and perform Z-axis accuracy detection. What's more, the table deviates from the original position towards the X direction, and the X-direction offset phantom image is acquired, and an X-axis accuracy detection is performed. Further, the table deviates from the original position towards the Y direction to acquire the Y-direction offset phantom image, and a Y-axis accuracy detection is performed. Then the table is moved to the original position, and the phantom images corresponding to the QA phantom at various gantry rotation angles and square field images formed by a beam-limiting system at various gantry rotation angles are acquired. The machine isocenter measurement, i.e., a laser offset, is performed, and the QA result of each QA object is finally obtained.

It should be noted that for the specific limitations of the above steps, please refer to the specific limitations of the QA method for the radiotherapy device described above, which will not be described repeatedly hereinafter.

It should be understood that although the steps in the flowcharts involved in the above embodiments are shown in sequence as indicated by the arrows, these steps are not necessarily executed in the order indicated by the arrows. Unless explicitly stated in this article, there is no strict order restriction on the execution of these steps, and these steps may be executed in other orders. Moreover, at least some of the steps in the flowcharts involved in the above embodiments may include multiple steps or multiple stages. These steps or stages are not necessarily executed at the same time, but may be executed at different times. The execution order of these steps or stages is not necessarily sequential, but may be performed in turn or alternately with other steps or at least part of the steps or stages in other steps.

Based on the same inventive concept, embodiments of the present disclosure also provide a QA device for a radiotherapy device used to implement the above-mentioned QA method for the radiotherapy device. The solutions to the problem provided by this device are similar to the solutions described in the above method. Therefore, the specific limitations in the embodiments of one or more QA devices of the radiotherapy device provided below may be found in the description of the QA device for the radiotherapy device, which will not be described repeatedly herein.

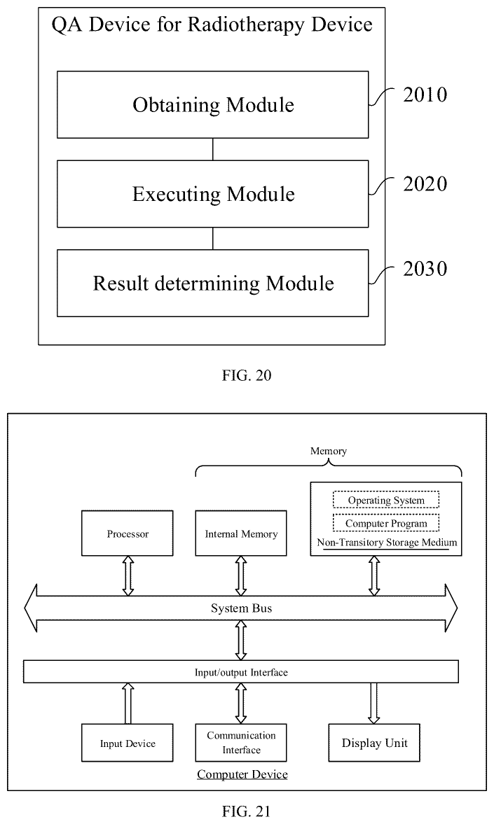

In one of the embodiments, as shown in FIG. 20, a QA device for a radiotherapy device is provided, and includes an obtaining module 2010 and a result determining module 2030.

The obtaining module 2010 is configured to obtain an image sequence based on the acquirement of EPID. The image sequence includes one or more correction images configured to correct the EPID and one or more portal images of a QA object.

The result determining module 2030 is configured to determine a QA result of the QA object of the radiotherapy device, according to a calibration parameter corresponding to the one or more correction image, and according to the one or more portal images.

In one of the embodiments, the obtaining module 2010 is further configured to obtain a QA plan sequence of the radiotherapy device. The QA plan sequence includes a QA plan for the QA object of the radiotherapy device. The QA device for the radiotherapy device further includes an executing module 2020. The executing module 2020 is configured to execute the QA plan for the QA object.

In one of the embodiments, the calibration parameter includes a pose calibration parameter corresponding to the EPID. The result determining module 2030 is specifically configured to calibrate the one or more portal images according to the pose calibration parameter, to obtain a target portal image of the QA object, and configured to determine the current measurement data corresponding to the QA object based on the image features of the target portal image.

In one of the embodiments, when the QA object includes an isocenter, the target portal images corresponding to the isocenter measurement include phantom images corresponding to the QA phantom at each of the preset gantry rotation angles, and square field images formed by a beam-limiting system at each of the gantry rotation angles. The QA phantom includes multiple mark points. The result determining module 2030 is specifically configured to determine a projection matrix corresponding to each gantry rotation angle according to mark-point spatial coordinates of the multiple mark points at initial positions and according to the mark-point projection coordinates of multiple mark points in each phantom image, configured to determine a straight-line trail of the radiation beam axis in a spatial coordinate system corresponding to the radiation beam axis at each gantry rotation angle, according to the projection matrix corresponding to each gantry rotation angle, and according to the projection coordinates of the radiation field center corresponding to the radiation field center in the square field image under each gantry rotation angle. The straight-line trail of the radiation beam axis is configured to determine a candidate mark point set. And the result determining module 2030 is configured to determine the radiation isocenter parameter corresponding to the isocenter according to a diameter of the target mark point.

In one of the embodiments, when the QA object includes a table, the target portal images corresponding to the table include a first initial phantom image and first post-movement phantom images of the QA phantom. The first post-movement phantom images include a phantom image corresponding to the direction of each coordinate axis of the spatial coordinate system. The QA phantom includes multiple mark points. The result determining module 2030 is specifically configured to determine post-movement mark-point projection coordinates of multiple mark points on coordinate axes according to each first post-movement phantom image, and configured to determine the post-movement mark-point spatial coordinates of the multiple mark points in coordinate axes according to a projection matrix corresponding to a first gantry rotation angle and the post-movement mark-point projection coordinates, and configured to determine actual movement parameters of the table in coordinate axes respectively according to the post-movement mark-point spatial coordinates of the multiple mark points in each coordinate axis, and according to a mapping relationship among the mark-point spatial coordinates of the multiple mark points at the first gantry rotation angle. The actual movement parameters are used to determine the current measurement data corresponding to the table.

In one of the embodiments, when the QA object includes a gantry, the target portal image of the gantry includes a second initial phantom image and second post-movement phantom images of the QA phantom. The second post-movement phantom images include phantom images at different heights of the table. The QA phantom includes multiple mark points. The result determining module 2030 is specifically configured to, for the same mark point, determine target mark-point projection coordinates in the second initial phantom image and in each of the second post-movement phantom images, and configured to superimpose each of the second post-movement phantom images and the second initial phantom image to obtain the superimposed phantom image, and configured to determine a connection line connecting the target mark-point projection coordinates in the superimposed phantom image corresponding to the same mark point to be a projection-position scaling path corresponding to the mark point, and configured to determine an intersection point of the projection-position scaling paths corresponding to all mark points to obtain the current measurement data corresponding to the gantry.

In one of the embodiments, the result determining module 2030 is specifically configured to determine the intersection-point projection coordinates of the intersection point, and configured to determine isocenter projection coordinates of the isocenter according to the projection matrix corresponding to the second gantry rotation angle and the spatial coordinates of the isocenter in the spatial coordinate system. The isocenter spatial coordinates are coordinates of the target mark point in the spatial coordinate system. The result determining module 2030 is further configured to determine the current measurement data corresponding to the gantry based on a target distance between the isocenter projection coordinates and the intersection-point projection coordinates.

In one of the embodiments, the QA object of the radiotherapy device includes an MLC. The correction images include a correction open filed image, a correction comb-shaped beam image, and a correction square field image. The correction square field image includes the correction square field rotation images under preset collimator angles respectively. The QA device for the radiotherapy device further includes a calibration parameter obtaining module. The calibration parameter obtaining module is configured to calibrate the correction open filed image to obtain a first calibration parameter, which includes, for example, coordinates or distribution of a bad pixel corresponding to a bad pixel correction, or response degree of each pixel to the same dose levels corresponding to a response correction; and configured, according to leaf description parameters of the leaves of the MLC in the correction comb-shaped beam image, to determine a second calibration parameter, which includes, for example, each central pixel row where each leaf center is located, a rotation angle of the EPID around each axis, and a scaling coefficient of the EPID; and configured to fit a radiation field center corresponding to the correction square field rotation image at each preset collimator angle, to obtain the third calibration parameter corresponding to the EPID, which, for example, includes the corresponding position of the collimator rotation center on the plate detector of the EPID; and configured to determine the pose calibration parameter corresponding to the correction images, based on the first calibration parameter, the second calibration parameter and the third calibration parameter corresponding to the EPID.

The above modules of the QA device for the radiotherapy device may be realized in whole or in part by software, hardware and combinations thereof. Each of the above modules may be embedded in or independent of the processor of the computer device in the form of hardware, or may be stored in the memory of the computer device in the form of software, so that the processor may call and execute the operations corresponding to the above modules.

In one of the embodiments, a computer device is provided. The computer device may be a terminal, and its internal structure diagram may be as shown in FIG. 21. The computer device includes a processor, memory, input/output interface, communication interface, display unit and input device. The processor, memory and input/output interface are connected through the system bus, and the communication interface, display unit and input device are connected to the system bus through the input/output interface. The processor of the computer device is configured to provide computing and control capabilities. The memory of the computer device includes nonvolatile storage media and internal memory. The nonvolatile storage medium stores operating systems and computer programs. This internal memory provides an environment for the execution of operating systems and computer programs in nonvolatile storage media. The input/output interface of the computer device is configured to exchange information between the processor and external devices. The communication interface of the computer device is used for wired or wireless communication with external terminals. The wireless mode may be implemented through WIFI, mobile cellular network, NFC (Near Field Communication) or other technologies. The computer program, when executed by the processor, implements a QA method for a radiotherapy device. The display unit of the computer device is used to form a visually visible picture, and may be a display screen, a projection device, or a virtual reality imaging device. The display screen may be a liquid crystal display or an electronic ink display. The input device of the computer device may be a touch layer covering the display screen, or it may be a button, trackball or touch pad provided on the computer device casing, or it may be an external keyboard, trackpad, or mouse, etc.

Those skilled in the art may understand that the structure shown in FIG. 21 is only a block diagram showing a partial structure related to the solutions of the present application, and does not constitute a limitation on the computer equipment to which the solutions of the present application is applied. A specific computer equipment may include more or fewer parts than those shown in the figure, or combine certain parts, or have a different arrangement of parts shown in the figure.

In one of the embodiments, a computer device is further provided, and the computer device includes a memory and a processor. A computer program is stored in the memory. When executing the computer program, the processor implements the steps in the above method embodiments.

In one of the embodiments, a non-transitory computer-readable storage medium is provided. The non-transitory computer-readable storage medium has a computer program stored thereon, and the computer program, when being executed by the processor, causes the processor to perform the steps of the above method.

In one of the embodiments, a computer program product is provided, and the computer program product includes executable instructions. When executed by a processor, the executable instructions implement the steps in each of the above method embodiments.

It should be noted that the user information (including but not limited to user equipment information, user personal information, etc.) and data (including but not limited to data used for analysis, stored data, displayed data, etc.) involved in the present disclosure are all information and data authorized by the user or fully authorized by all parties, and the collection, use and processing of relevant data must comply with the relevant laws, regulations and standards of relevant countries and regions.

Those ordinary skilled in the art may understand that all or part of the process in the method of the above embodiments may be implemented by instructing the relevant hardware through executable instructions, and the executable instructions may be stored in a non-transitory computer-readable storage medium. The executable instructions, when being executed, may include the processes of the embodiments of the methods above. Where, any reference to memory, storage, database, or other media used in the various embodiments provided in this application may include at least one of non-transitory and transitory memory. Non-transitory memory may include read-only memory (ROM), magnetic tape, floppy disk, flash memory, or optical memory, high-density embedded non-transitory memory, resistance random access memory (ReRAM), magneto resistive random-access memory (MRAM), ferroelectric random-access memory (FRAM), phase change memory (PCM), graphene memory, and the like. The transitory memory may include random access memory (RAM) or external cache memory. By way of illustration and not limitation, the RAM may be in various forms, such as static random-access memory (SRAM) or dynamic random-access memory (DRAM), etc. The databases involved in the embodiments provided in the present application may include at least one of a relational database and a non-relational database. The non-relational databases may include, but are not limited to, a blockchain-based distributed database, and the like. The processors involved in the embodiments provided in the present application may be, but are not limited to, a general-purpose processor, a central processing unit, a graphics processor, a digital signal processor, a programmable logic, a quantum-computing-based data processing logic, and the like.

The technical features of the embodiments above may be combined arbitrarily. To make the description concise, not all possible combinations of the technical features in the above embodiments are described. However, as long as there are no contradictions in the combinations of these technical features, all of the combinations should be considered to be within the scope of the specification.

The embodiments above only represent several implementation modes of the present application, and the description thereof is relatively specific and detailed, but it should not be construed as limiting the scope of the patent. It should be noted that for those skilled in the art, various modifications and improvements may be made without departing from the concept of the present application, and all these modifications and improvements belong to the protection scope of the present application. Therefore, the scope of protection of the patent application should be subject to the appended claims.

1. A quality assurance (QA) method for a radiotherapy device, the radiotherapy device comprising an electronic portal imaging device (EPID), wherein the QA method comprises:
obtaining an image sequence based on acquirement of EPID, the image sequence comprising one or more correction images for correcting the EPID, and one or more portal images of a QA object of the radiotherapy device; and
determining a QA result of the QA object, according to the one or more portal images and a calibration parameter corresponding to the one or more correction images.

2. The QA method according to claim 1, wherein before obtaining the image sequence based on the acquirement of the EPID, the QA method further comprises:
obtaining a QA plan sequence for the radiotherapy device, the QA plan sequence comprising a QA plan for the QA object; and
executing the QA plan.

3. The QA method according to claim 2, wherein the QA plan comprises setting a shape of a radiation field and a position of a QA phantom.

4. The QA method according to claim 1, wherein determining the QA result of the QA object according to the one or more portal images and the calibration parameter corresponding to the one or more correction images, comprises:
determining current measurement data corresponding to the QA object, according to the one or more portal images and the calibration parameter corresponding to the one or more correction images; and
determining the QA result of the QA object based on the current measurement data corresponding to the QA object.

5. The QA method according to claim 4, wherein:
the calibration parameter comprises a pose calibration parameter corresponding to the EPID; and
determining the current measurement data corresponding to the QA object according to the one or more portal images, and the calibration parameter corresponding to the one or more correction images, comprises:
calibrating the one or more portal images according to the pose calibration parameter, to obtain a target portal image of the QA object; and
determining the current measurement data corresponding to the QA object, based on image features of the target portal image.

6. The QA method according to claim 5, wherein:
the QA object comprises an isocenter, and the target portal image corresponding to an isocenter measurement comprises a phantom image of a QA phantom at each preset gantry rotation angle and a square field image formed by a beam-limiting system at each preset gantry rotation angle, or a phantom image of a QA phantom in a corresponding field at each preset gantry rotation angle;
wherein the QA phantom comprises multiple mark points; and
determining the current measurement data corresponding to the QA object based on the image features of the target portal image, comprises:
determining a projection matrix corresponding to each preset gantry rotation angle according to mark-point spatial coordinates of the multiple mark points at initial positions, and according to mark-point projection coordinates of the multiple mark points in each phantom image;
determining a straight-line trail of a radiation beam axis in a spatial coordinate system corresponding to the radiation beam axis at each preset gantry rotation angle according to the projection matrix corresponding to each preset gantry rotation angle, and according to projection coordinates of a radiation field center corresponding to the radiation field center in the square field image under each preset gantry rotation angle; and
determining a radiation isocenter parameter corresponding to the isocenter according to an intersection point of straight-line trails of radiation beam axes.

7. The QA method according to claim 5, wherein:
the QA object comprises a table, and the target portal image corresponding to the table comprises a first initial phantom image and first post-movement phantom images acquired at a gantry rotation angle, and the first post-movement phantom images comprise phantom images each corresponding to a direction of each coordinate axis of a spatial coordinate system;
wherein the QA phantom comprises multiple mark points; and
determining the current measurement data corresponding to the QA object based on the image features of the target portal image, comprises:
determining post-movement mark-point projection coordinates of the multiple mark points in coordinate axes according to each of the first post-movement phantom images; and
determining actual movement parameters of the table in the coordinate axes respectively corresponding to the gantry angle and the post-movement mark-point projection coordinates.

8. The QA method according to claim 5, wherein:
the QA object comprises a gantry, the target portal image corresponding to the gantry comprises a second initial phantom image and second post-movement phantom images of the QA phantom, and the second post-movement phantom image comprises phantom images at different heights of the table;
the QA phantom comprises multiple mark points; and
determining the current measurement data corresponding to the QA object according to the image features of the target portal image, comprises: determining, for the same mark point, target mark-point projection coordinates in the second initial phantom image and in each of the second post-movement phantom images to obtain the current measurement data corresponding to the gantry.

9. The QA method according to claim 5, wherein:
the QA object comprises a beam component, and the target portal image corresponding to the beam component comprises a preset number of square field images; and
determining the current measurement data corresponding to the QA object based on the image features of the target portal image, comprises:
superimposing the preset number of square field images to obtain a superimposed square field image;
translating the superimposed square field image and
adjusting the translated square field image according to an image scaling coefficient, to obtain the current measurement data corresponding to the beam component.

10. The QA method according to claim 5, wherein:
the QA object comprises a collimator, and the target portal image corresponding to the collimator comprises a first correction square field rotation image corresponding to a first collimator angle and a second correction square field rotation image corresponding to a second collimator angle; and
determining the current measurement data corresponding to the QA object according to the image features of the target portal image, comprises:
detecting a boundary of a jaw according to the first correction square field rotation image and the second correction square field rotation image, to obtain a first boundary line and a second boundary line;
determining a first boundary measurement inclination angle and a second boundary measurement inclination angle according to the first boundary line and the second boundary line; and
obtaining the current measurement data corresponding to the collimator according to the first boundary measurement inclination angle and the second boundary measurement inclination angle.

11. The QA method according to claim 5, wherein:
the image sequence comprising a plurality of correction images;
the plurality of correction images comprise a correction open filed image, a correction comb-shaped beam image, and a correction square field image;
the correction square field image comprises a correction square field rotation image at each preset collimator angle; and
before determining the QA result of the QA object according to the one or more portal images and the calibration parameter corresponding to the one or more correction images, the method further comprises:
calibrating the correction open filed image to obtain a first calibration parameter;
determining a second calibration parameter based on leaf description parameters corresponding to leaves of a multi-leaf collimator (MLC) in the correction comb-shaped beam image;
fitting a radiation field center corresponding to the correction square field rotation image at each preset collimator angle to obtain a third calibration parameter; and
determining a pose calibration parameter corresponding to the plurality of correction images based on the first calibration parameter, the second calibration parameter and the third calibration parameter.

12. The QA method according to claim 14, wherein:
the QA object comprises the MLC, and the target portal image corresponding to the MLC comprises a collimator portal image, and the collimator portal image is an image acquired after a shape of the radiation field of the MLC is configured to have a first size; and
determining the current measurement data corresponding to the QA object according to the image features of the target portal image, comprises:
performing a transverse gradient transformation on the collimator portal image, and determining a position of a pixel with the largest gray gradient change corresponding to each leaf in the collimator portal image, on which a transverse gradient transformation is performed, as a pixel position to be converted corresponding to each leaf, and
converting each pixel position to be converted into a radiation field position in a radiation field coordinate system, to obtain a current measurement position of each leaf, and using the current measurement position of each leaf as the current measurement data corresponding to the MLC.

13. The QA method according to claim 5, wherein:
the QA object comprises a jaw, and the target portal image corresponding to the jaw comprises a jaw beam image, and the jaw beam image is an image acquired after a shape of the radiation field of the jaw is configured to have a second size; and
determining the current measurement data corresponding to the QA object according to the image features of the target portal image, comprises:
performing a longitudinal gradient transformation on the jaw beam image, determining a jaw pixel position, and obtaining a longitudinal jaw measurement position according to the jaw pixel position;
performing a transverse analysis on the jaw beam image to obtain a transverse jaw measurement position; and
using the longitudinal jaw measurement position and the transverse jaw measurement position as the current measurement data corresponding to the jaw.

14. The QA method according to claim 11, wherein:
the QA object comprises a plate detector; and
determining the current measurement data corresponding to the QA object according to the image features of the target portal image, comprises:
determining a current measurement position of the collimator rotation center in a plate coordinate system, wherein the plate coordinate system is a pixel coordinate system corresponding to the plate detector;
determining a leaf lateral width of the MLC based on the correction comb-shaped beam image, and determining a distance from the plate detector to an X-ray source of the radiotherapy device based on the leaf lateral width as a current measured SID; and
obtaining the current measurement data corresponding to the plate detector based on the current measurement position of the collimator rotation center in the plate coordinate system and based on the current measured SID.

15. A QA method for a radiotherapy device, the radiotherapy device comprising an electronic portal imaging device (EPID), wherein the QA method comprises:
determining a calibration parameter corresponding to one or more correction images based on the one or more correction images acquired by the EPID for correcting the EPID;
obtaining a target portal image of a QA object, based on the calibration parameter and one or more portal images of the QA object acquired by the EPID; and
determining a QA result of the QA object of the radiotherapy device according to the target portal image.

16. A computer device, comprising a memory and a processor, the memory storing a computer program, wherein when the computer program is executed, the processor is configured to implement steps of the QA method of claim 1.

17. A non-transitory computer-readable storage medium, having executable instructions stored thereon, wherein, the executable instructions, when executed by a processor, are configured to cause the processor to implement steps of the QA method of claim 1.

18. A computer program product, comprising executable instructions, wherein the executable instructions, when executed by a processor, implement steps of the method of claim 1.
